(19) United States
(12) Patent Application Publication
(10) Pub. No.: US 2022/0397564 A1
(43) Pub. Date: Dec. 15, 2022

(54) ENGINEERING SPATIAL-ORGANIZED CARDIAC ORGANOIDS FOR DEVELOPMENTAL TOXICITY TESTING

(71) Applicants: Zhen Ma, Jamesville, NY (US); Plansky Hoang, Syracuse, NY (US)

(72) Inventors: Zhen Ma, Jamesville, NY (US); Plansky Hoang, Syracuse, NY (US)

(73) Assignee: SYRACUSE UNIVERSITY, SYRACUSE, NY (US)

(21) Appl. No.: 17/837,697

(22) Filed: Jun. 10, 2022

Related U.S. Application Data
(60) Provisional application No. 63/209,501, filed on Jun. 11, 2021.

(51) Int. Cl. G01N 33/50; C12N 5/077

(52) U.S. Cl. CPC G01N 33/5014; C12N 5/0657; G01N 33/5044; C12N 2506/45; C12N 2535/10

(57) ABSTRACT

A developmental toxicity screening assay using spatially organized cardiac organoids with contracting cardiomyocytes in the center surrounded by stromal cells distributed along the pattern perimeter engineered from human induced pluripotent stem cells (hiPSCs). Cardiac organoids generated from 600 μm-diameter circles were used as a developmental toxicity screening assay for the quantification of the embryotoxic potential of nine pharmaceutical compounds. The cardiac organoids were demonstrated as having a potential use as an in vitro platform for studying organoid structure-function relationships, developmental processes, and drug-induced cardiac developmental toxicity.

### CROSS-REFERENCE TO RELATED APPLICATIONS

The present application claims priority to U.S. Provisional Application No. 63/209,501, filed on Jun. 11, 2021.

### STATEMENT REGARDING FEDERALLY SPONSORED RESEARCH AND DEVELOPMENT

This invention was made with government support under Grant No. R01HD101130 awarded by the National Institutes of Health (NIH). The government has certain rights in the invention.

### BACKGROUND OF THE INVENTION

### 1. Field of the Invention

The present invention relates to toxicity screening assays and, more specifically, to the use spatially organized cardiac organoids engineered from human induced pluripotent stem cells to evaluate potential toxicity.

### 2. Description of the Related Art

Recent progress in stem cell-based organoid technology offers unique opportunities to in vitro recapitulate biological processes of organogenesis into spatially organized tissue structures that resemble the architecture and functions of specific tissues. Integration of organoid technology and microfabrication has provided promising ways to guide self-organization and spatial pattern formation of developing biological tissues. Microfluidic systems could precisely control the localization of morphogen source and gradient to guide the spatial human pluripotent stem cells (hPSC) differentiation and organization into in vitro synthetic embryonic tissues, such as in germ layer patterning, as well as amniotic and epiblast layer separation. Surface micropatterning techniques were also able to provide geometric confinement for modeling gastrulation process, where PSCs were patterned and differentiated to form concentric rings indicative of specific germ layers. These examples illustrate the critical need for spatiotemporal engineering of cell microenvironments to guide the structure and function of stem cell organoids to specific biological tissues.

Despite extensive efforts in controlling stem cell lineage specification via biophysical inputs, there are few studies focusing on how 2D patterned cell colonies could give rise to organoids with defined structure-function relationships. Understanding these relationships require comprehensive analysis of multiple variables simultaneously, which increases the data dimensionality for analysis and visualization. As methods to detect cardiac functions evolve and become more sophisticated, large-scale multidimensional data requires more advanced analytics to effectively comprehend the functional outcomes. Currently, data dimensionality reduction techniques, which are the basis of bioinformatics analysis, are still underexplored for analyzing tissue level structural and functional properties. The combination of tissue engineering, organoid technology and advanced data mining techniques would potentially provide the versatility and capability to discover trends and relationships to guide new engineering designs with a spectrum of biological structures and functions.

hPSC-derived organoids exhibit characteristics of specific tissue lineages at their early developmental stages, thus providing great potential as in vitro assays of developmental drug toxicity. In vivo animal models and in vitro mouse embryonic stem cell tests (mEST) are widely implemented by pharmaceutical companies as biological assays for embryotoxicity screening. To overcome species barriers that impose limitations in traditional drug screening, hPSC technology has been proposed to replace the mEST for better predictions of human-specific developmental toxicity. However, most 2D stem cell-based assays lack the capability of morphological scoring of 3D tissue morphogenesis. This undermines the predictability of drug-induced teratogenicity, which potentially leads to structural malformations manifested in late prenatal fetus development. Moreover, traditional organoid technology exhibits relatively random positioning of tissue regions of specific cell types, and these regions are not reliably spatial-organized relative to one another. Heterogeneity in organoid formation also makes it difficult for embryotoxicity drug testing purposes with high consistency and reproducibility.

The majority of in vitro cardiac tissue models focus on accurate recapitulation of physiologically relevant tissue structures of adult human heart, which are generally achieved by populating pre-fabricated 3D biomaterial scaffolds with pre-differentiated hiPSC-derived cardiomyocytes (hiPSC-CMs). These adult-mimicking model systems are designed to enhance the maturity of hiPSC-CMs for the purpose of drug screening and disease modeling, but not designed for studying dynamic cellular self-organization occurring along with the cardiac differentiation process. In contrast, stem cell-derived organoids are designed to resemble the early developing organs through self-organization of differentiating cells into spatial-distinct tissue-specific structures. However, cardiac organoids are still largely generated by aggregating pre-differentiated hiPSC-CMs with other stromal cells, instead of originating from directed stem cell differentiation.

According there is a need in the art for approaches that create and use cardiac organoids that reflect the biological process of tissue self-assembly and morphogenesis during early heart formation for screening of drug candidates for potential drug-induced developmental toxicity.

### BRIEF SUMMARY OF THE INVENTION

The present invention comprises the formation and use of optimized cardiac organoids from 2D micropatterned human induced PSCs (hiPSCs). Organoids generated from genome engineered GCaMP6f hiPSCs were used for integrated functional analysis of calcium transient and contraction motion. Using data mining tools, relationships amongst a multitude of organoid metrics of tissue structure and contractile functions were established. The inter-dependency amongst these parameters associated with the geometric sizes was explored, which highlighted that biophysical microenvironment could modulate tissue structure and cardiac function. The model was then evaluated as a reliable indicator of cardiac developmental toxicity by quantifying the drug effects based on cardiac differentiation, contractile behaviors and 3D tissue morphology. More specifically, the present invention comprises a method for screening a target compound for embryotoxicity that includes the step of forming an amount of cardiac organoids, each of which includes a contracting cardiomyocyte surrounded by a plurality of stromal cells distributed therearound, from a quantity of human induced pluripotent stem cells. The human induced pluripotent stem cells are exposed to the target compounds during the step of forming the amount of spatially organized cardiac organoids. Finally, the amount of cardiac differentiation, the contractile behavior, and the three dimensional tissue morphology of the amount of cardiac organoids are considered after exposing the amount of spatially organized cardiac organoids to the target compounds to determine whether the target compound is embryotoxic. The cardiac organoids are spatially organized and can be formed using a micropatterned substrate. The micropatterned substrate may include a plurality of circles, each which has a diameter of about 600 μm. The human induced pluripotent stem cells are exposed to the target compounds during the step of forming the amount of spatially organized cardiac organoids comprises exposing the human induced pluripotent stem cells on a first day of differentiation. The step of forming the amount of cardiac organoids may include the step of differentiating human induced pluripotent stem cells by small molecule modulation of the Wnt/β-catenin pathway. The step of forming the amount of cardiac organoids includes the step of oxygen plasma etching an amount of poly(ethylene glycol) to form the micropatterned substrate. The step of oxygen plasma etching the amount of poly(ethylene glycol) includes using a mask formed from poly(dimethyl siloxane). The step of forming the amount of cardiac organoids includes the step of coating the micropatterned surface were coated with a diluted hESC-qualified matrix. The step of forming the amount of cardiac organoids includes the step of differentiating the human induced pluripotent stem cells with a GSK3 inhibitor.

The results of drug-induced cardiac developmental toxicity evaluation on hiPSC-based cardiac organoids using the present invention was also compared and confirmed using the effects on in vivo cardiac development using whole embryo culture (WEC) of living Danio rerio (zebrafish) embryos. The human cardiac organoid model and approach of the present invention thus provides as a versatile platform to assess cardiac organogenesis and developmental toxicity, which can be adopted for pharmaceutical development and fetal safety assessments.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1A is a series of schematics showing human iPSC micropatterning and differentiation into cardiac organoids showing the procedure for micropatterning hiPSCs on standard tissue culture plate using a selective PEG-based etching method.

FIG. 1B is bright-field microscopy images of arrays of micropatterned hiPSCs with different sizes and shapes. Scale bar, 400 μm.

FIG. 1C is spatial-organized cardiac organoids showed cardiac muscle on the top center with cardiomyocyte-specific proteins (cardiac troponin T (cTnT), β myosin heavy chain (MHC-β) sarcomeric α-actinin and cardiac troponin I (cTnI)) and smooth muscle-like cells on the perimeter of the organoids with stromal cell markers (smooth muscle actin (α-SMA), calponin, transgelin (α-SM22), and vimentin from 600 μm organoids. Scale bar, 200 μm.

FIG. 1D are maximum intensity confocal projections of cardiomyocytes on the cardiac organoids. Scale bar, 50 μm.

FIG. 2A is a schematic of cardiac differentiation timeline to generate cardiac organoids.

FIG. 2B is a series of images of patterned cell morphology changes from 2D monolayer of cells (DO) into 3D tissue (D6 onward) throughout differentiation; scale bars 600 μm.

FIG. 2C is a series of images of patterned hiPSCs retain expression of pluripotent markers when confluent (From left to right: OCT4, NANOG, ECAD, SOX2, SSEA4.

FIG. 2D is an image 24 h after CHIR treatment, patterned hiPSCs express mesodermal marker brachyury.

FIG. 2E is an image on Day 8 of differentiation, cardiac organoids express ISL1.

FIG. 2F is an image of NKX2.5.

FIG. 2G is an imager of GATA4 on Day 10, indicative of early cardiac progenitor cells.

FIG. 2H is a graph of stromal gene expression illustrates higher upregulation of stromal markers in organoids relative to 2D monolayer differentiation and higher upregulation of stromal cell makers in 200-μm organoids relative to 600-μm and 1000-μm organoids. DCt values were calculated relative to the average Ct of GAPDH and 18S housekeeping controls. Scale bars 200 μm;

FIG. 3A is a graph showing contraction function analysis of cardiac organoids generated from GCaMP6f hiPSCs. Videos of beating organoids were characterized with using motion tracking analysis to generate a motion waveform where each double-peak represents a contraction-relaxation cycle. The Peak-to-peak interval describes the time between the contraction and relaxation peaks.

FIG. 3B is a graph of transient calcium flux signals were acquired from capturing videos of GCaMP6f cardiac organoids and plotting z-axis profiles using ImageJ. Fluorescence bleaching (descending blue signal) was corrected (red signal) using in-house MATLAB scripts. Time decay parameters τ0, τ50, and τ75 were acquired by measuring time intervals from the signal initiation to the maximum calcium flux (τ0), for the maximum flux to decay to 50% (τ50), and for the maximum flux to decay to 25% (τ75). All figures represent schematic illustrations of characterization parameters.

FIG. 3C is a graphs of raw data metrics used for data mining and tSNE clustering of organoid contraction functions. Individual points for each variable are shown to illustrate sample distribution. Comparable trends are seen where small patterns significantly prolong the beat duration, based on metrics t75, t50, peak-to-peak interval and pulse duration. This can be correlated to the area ratio, which is also significantly greater in small 200 μm organoids. Moreover, most variables illustrated some degree of pattern size dependency, with the most significant functional variations seen in 200 μm organoids. All statistics analyzed using ANOVA with Tukey multiple comparison tests. p£0.05 is considered significant (*).

FIG. 4A is a series images showing pattern size effects on cardiac organoid characteristics where cardiac organoids generated from the circular patterns with different sizes (200 μm-1000 μm in diameter) showed spatial-organization of cardiomyocytes and stromal cells and 3D dome-shape in the X-Z plane. Scale bar, 200 μm.

FIG. 4B is a graph of the area ratio between cardiomyocyte staining and entire pattern size showed larger coverage but higher variation in cardiac muscle differentiation on the cardiac organoids of 200 μm, 800 μm and 1000 μm patterns (n=9, *p≤0.0001 between 200 μm and 400/600 μm; *p≤0.0001 between 400/600 and 800/1000 μm).

FIG. 4C is a graph showing that the cardiac organoids of 600 μm patterns exhibited better 3D morphology with largest values in height (n=8, *p=0.0126 between 200 μm and 600 μm; *p=0.0126 between 600 and 800/1000 μm).

FIG. 4D is a graph showing FWHM (n=8, *p≤0.0001 between 600 μm and 200/400 μm; *p≤0.0001 between 600 and 800/1000 μm).

FIG. 4E is a graph of the contractile function parameters used to cluster individual organoids with different pattern sizes and generate a t-SNE plot to evaluate organoid-to-organoid correlations.

FIG. 4F is a graph of the t-SNE gradients plotted for beat rate.

FIG. 4G is a graph of the t-SNE gradients plotted for maximum contraction velocity.

FIG. 4H is a graph of the t-SNE gradients plotted for τ75.

FIG. 4I is a graph of the t-SNE gradients plotted for area ratio.

FIG. 4J is a heat map illustrating all contractile functions parameters relative to pattern size showed strong correlation between size and prolonged contraction duration, especially for the organoids of small patterns.

FIG. 4K is a correlation plot illustrating proportional relationships between contraction duration parameters, but inverse correlations between contraction duration and contraction rate. All box plots show the minimum, maximum, median, and 25th and 75th percentiles, and statistical analysis was performed based on analysis of variance (ANOVA) with Tukey's multiple comparison test.

FIG. 5A is a graph showing t-SNE gradient plots of contraction functions of cardiac organoids. Contraction function was plotted using color gradients to illustrate the influence of organoid size on relaxation velocity.

FIG. 5B is a graph showing t-SNE gradient plots of contraction functions of cardiac organoids. Contraction function was plotted using color gradients to illustrate the influence of organoid size on τ50,

FIG. 5C is a graph showing t-SNE gradient plots of contraction functions of cardiac organoids. Contraction function was plotted using color gradients to illustrate the influence of organoid size on peak-interval,

FIG. 5D is a graph showing t-SNE gradient plots of contraction functions of cardiac organoids. Contraction function was plotted using color gradients to illustrate the influence of organoid size on pulse duration,

FIG. 5E is a graph showing t-SNE gradient plots of contraction functions of cardiac organoids. Contraction function was plotted using color gradients to illustrate the influence of organoid size on τ0, and

FIG. 5F is a graph showing t-SNE gradient plots of contraction functions of cardiac organoids. Contraction function was plotted using color gradients to illustrate the influence of organoid size on maximum calcium flux.

FIG. 5G is a graph showing correlation coefficients showing individual variables correlation with other variable. A correlation coefficient closer to 1 is equivalent to positive correlation, where negative correlation is associated with values close to −1.

FIG. 5H is a graph showing the P-value representation of correlation coefficient significance. (*) denotes p£0.05; (**) denotes p£0.01; (***) denotes p£0.001

FIG. 6A is a series of graphs showing developmental toxicity assay of cardiac organoids in response to treatment with Category B and C drugs, where amoxicillin showed moderate toxicity with decreased beat rate (ANOVA, n≥11, *p=0.0215) and increased beat duration (ANOVA, n≥11, *p=0.0484) at high concentrations. Amoxicillin treatment at low concentrations, however, produced smaller cardiac tissues in area ratio (ANOVA, n≥18, *p=0.0003 between Controls and 1 μM), height (ANOVA, n≥7, *p=0.0006), and FWHM (ANOVA, n≥16, *p≤0.0001).

FIG. 6B is a series of graphs showing developmental toxicity assay of cardiac organoids in response to treatment with Category C drug, Rifampicin. In all assays, cardiac organoids failed to differentiate at 100 μM treatment, and toxicity was also observed in contraction velocity (ANOVA, n≥12, *p<0.0001), beat rate (ANOVA, n≥12, *p<0.05), height (ANOVA, n≥10, *p≤0.05) and in FWHM (ANOVA, n≥21, *p≤0.05).

FIG. 7A is a series of graphs of developmental toxicity assay of cardiac organoids in response to treatment with Category D drugs. With doxycycline treatment, cardiac organoids failed to differentiate at 100 μM treatment, and did not form robust cardiac tissues at 10 μM. Significant toxicity was observed in contraction velocity (ANOVA, n≥7, *p<0.05), beat rate (ANOVA, n≥7, *p<0.05), beat duration (ANOVA, n≥7, *p<0.0001), and height (ANOVA, n=8, *p 0.05 relative to controls) and in FWHM (ANOVA, n≥21, *p<0.05).

FIG. 7B is a series of graphs showing that with lithium carbonate treatment, no significant toxicity effects were seen in any contraction function. However, moderate toxicity was seen in higher concentrations for the area ratio (ANOVA, n≥26, *p<0.0001) and in FWHM (ANOVA, n≥20, *p<0.0001) relative to the controls.

FIG. 7C is a series of graphs showing that phenytoin showed no contractile functions at 100 μM concentration, with moderate effects at 1 μM on beat rate (ANOVA, n≥11, *p<0.0001) and beat duration (ANOVA, n≥11, *p<0.0001). Organoids were also smaller in area ratio at 10 μM treatment (ANOVA, n=28, *p<0.0001) and smaller at all concentrations in height (ANOVA, n≥12, *p<0.0001) and at low and moderate concentrations in FWHM (ANOVA, n=20, *p<0.0001) relative to controls.

FIG. 8A is a series of graphs showing developmental toxicity assay of cardiac organoids in response to treatment with retinoids Tretinoin (Category D) and Isotretinoin (Category X). With tretinoin, cardiac tissue failed to differentiate at 10 μM concentrations with no contraction functions. Contraction velocity was lower at 0.1 μM (ANOVA, n≥7, *p<0.0001), whereas beat rate was faster at 0.1 μM and 1 μM (ANOVA, n≥7, *p<0.0001). Beat duration was significantly lower at 1 μM (ANOVA, n≥7, *p<0.0001) as well. Low concentration of 0.1 μM also showed toxic effects on area ratio (ANOVA, n≥12, *p<0.0001), height (ANOVA, n≥8, *p<0.0001), and FWHM (ANOVA, n≥12, *p<0.0001).

FIG. 8B is a series of graphs showing that cardiac tissue failed to develop at 1 μM and 10 μM concentrations with no contraction functions. Low concentration of 1 μM resulted in faster beat rate (ANOVA, n≥13, *p<0.0001) and smaller area ratio (ANOVA, n≥31, *p<0.0001). However, high concentrations produced organoids that were significantly taller in height (ANOVA, n≥9, *p<0.0001) and larger in FWHM (ANOVA, n≥12, *p<0.0001).

FIG. 8C is a series of representative images show cardiac tissue formation at low (0.1 μM) concentration, but a large mound with no cardiac tissue at high (10 μM) concentration of retinoid exposures. Scale bars 200 μm.

FIG. 9A shows zebrafish whole embryo culture (zWEC) assay of cardiac looping for seven drug compounds where morphological scoring was performed on heart structures of developing zebrafish embryos based on either normal (D-loop), reverse (L-loop), no loop, or no score where the heart did not express GFP and/or fall into the previous three categories.

FIG. 9B is a graph showing Category C rifampicin produced mild developmental toxic effect. A sample size of n≥19 embryos (pooled from 3 independent experiments) was used for all treatment groups.

FIG. 9C is a graph showing Category D phenytoin produced mild developmental toxic effect. A sample size of n≥19 embryos (pooled from 3 independent experiments) was used for all treatment groups.

FIG. 9D is a graph showing Category B amoxicillin displayed moderate developmental toxicity. A sample size of n≥19 embryos (pooled from 3 independent experiments) was used for all treatment groups.

FIG. 9E is a graph showing Category D lithium carbonate produced severe developmental toxic effects at all concentrations. A sample size of n≥19 embryos (pooled from 3 independent experiments) was used for all treatment groups.

FIG. 9F is a graph showing Category D doxycycline produced severe developmental toxic effects at all concentrations. A sample size of n≥19 embryos (pooled from 3 independent experiments) was used for all treatment groups.

FIG. 9G is a graph showing Category D tretinoin produced severe developmental toxic effects at all concentrations. A sample size of n≥19 embryos (pooled from 3 independent experiments) was used for all treatment groups.

FIG. 9H is a graph showing Category X isotretnoin produced severe developmental toxic effects at all concentrations. A sample size of n≥19 embryos (pooled from 3 independent experiments) was used for all treatment groups.

FIG. 10A is schematic showing a timeline of cardiac organoid generation and continuous drug exposure.

FIG. 10B are epi-fluorescent microscopy images of the cardiac organoids for comparison between untreated controls with doxylamine succinate (category A drug)

FIG. 10C are epi-fluorescent microscopy images of the cardiac organoids for comparison between untreated controls with thalidomide (category X drug).

FIG. 10D is a graph showing the area ratio of cardiac muscle coverage showed an increase with doxylamine succinate (n=29, *p≤0.0001).

FIG. 10E is a graph showing the area ratio of cardiac muscle coverage showed a decrease with thalidomide at high concentration (n≥28, *p≤0.0001).

FIG. 10F is a graph showing the contraction velocity of cardiac contractile motion showed no drug effect from doxylamine succinate.

FIG. 10G is a graph showing the contraction velocity of cardiac contractile motion showed no drug effect from or thalidomide.

FIG. 10H is a graph showing that the beat rate showed an increase with thalidomide at 1 μM and 10 μM concentrations (n≥12, *p≤0.0001).

FIG. 10I is a graph showing that the cardiac organoids under doxylamine succinate exposure showed no drug effects on the 3D organoid morphology at all the tested concentrations.

FIG. 10J is a graph showing that thalidomide exposure induced significant structural impairment on 3D organoid formation, showed as lower height (n≥9, *p≤0.0001) and FWHM (n≥14, *p≤0.0001) at high concentrations.

FIG. 10K are representative confocal projections of cardiac organoids at untreated control.

FIG. 10L is an image of a control for FIG. 10M.

FIG. 10M is an image illustrating that 100 μM thalidomide exposure showed severe abnormal organoid formation for quantitative morphological scoring.

FIG. 10N is an image and series of graphs illustrating that cardiac looping scored as a cardiac developmental toxicity evaluation in the zebrafish whole embryo culture assay (zWEC) showed no significant drug effect from doxylamine succinate exposure, and a very mild toxicity level from high dosage exposure of thalidomide. A sample size of n≥40 embryos were analyzed for these treatment groups. In all panels, box plots show the minimum, maximum, median, and 25th and 75th percentiles, and statistical analysis was performed based on analysis of variance (ANOVA) with Dunnett's multiple comparison test against controls.

FIG. 11A is a pair of graphs of 2D monolayer differentiation and organoid differentiation using WTC11 hiPSCs showing that over 50% cells are cTnT+ cardiomyocytes. Sample size≥4.

FIG. 11B is a pair of graphs showing that reproducibility was demonstrated using a second hiPSC line (Yale WT), which also resulted in over 50% of cells expressing cTnT. Sample size n=5.

FIG. 11C is a series of flow cytometry density plots quantifying cardiac differentiation efficiency of organoids treated with doxylamine succinate and thalidomide. Doxylamine succinate treatment concentrations did not affect the percentage of cells expressing cTnT. Thalidomide treatment caused a considerable reduction in cTnT positive cells with increasing concentration.

FIG. 12 is a series of graphs showing developmental toxicity assay of cardiac organoids in response to treatment with Category B drug. Amoxicillin showed moderate toxicity with decreased beat rate (ANOVA, n≥11, *p=0.0215) and increased beat duration (ANOVA,n≥11, *p=0.0484) at high concentrations. Amoxicillin treatment at low concentrations, however, produced smaller cardiac tissues in area ratio (ANOVA, n≥18, *p=0.0003 between Controls and 1 μM), height (ANOVA, n≥7, *p=0.0006), and FWHM (ANOVA, n≥16, *p<0.0001).

FIG. 13 is a series of graphs showing a developmental toxicity assay of cardiac organoids in response to treatment with Category C drug, Rifampicin. In all assays, cardiac organoids failed to differentiate at 100 μM treatment, and toxicity was also observed in contraction velocity (ANOVA, n≥12, *p<0.0001), beat rate (ANOVA, n≥12, *p<0.05), height (ANOVA, n≥10, *p<0.05) and in FWHM (ANOVA, n≥21, *p<0.05).

### DETAILED DESCRIPTION OF THE INVENTION

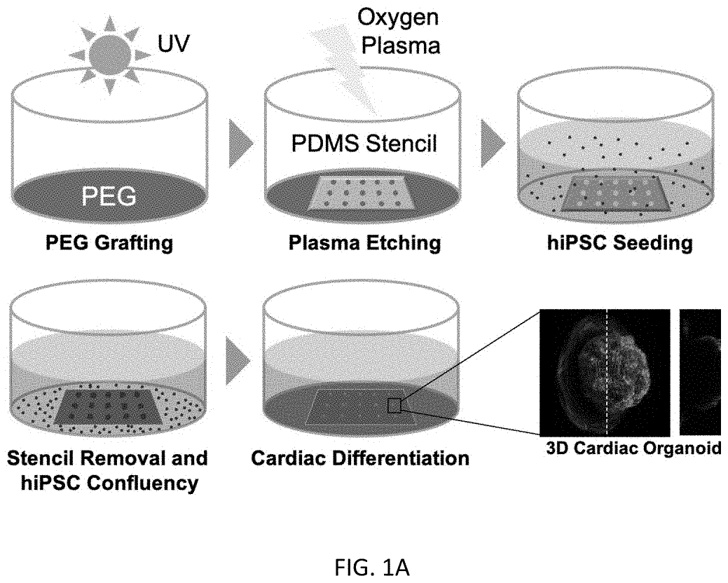

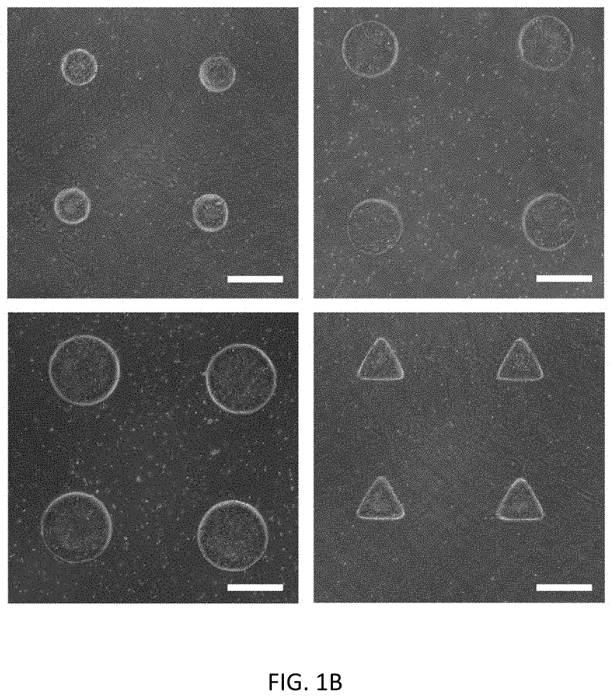

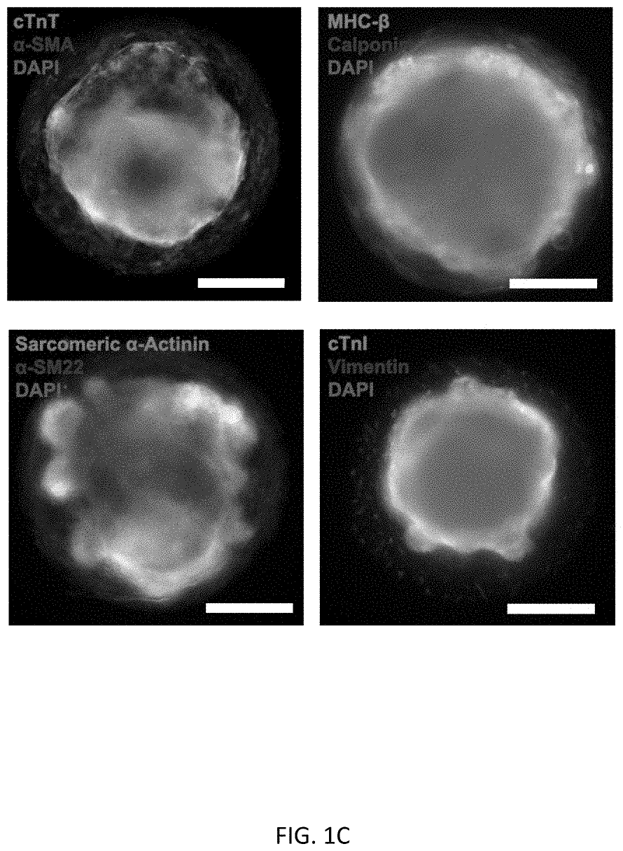

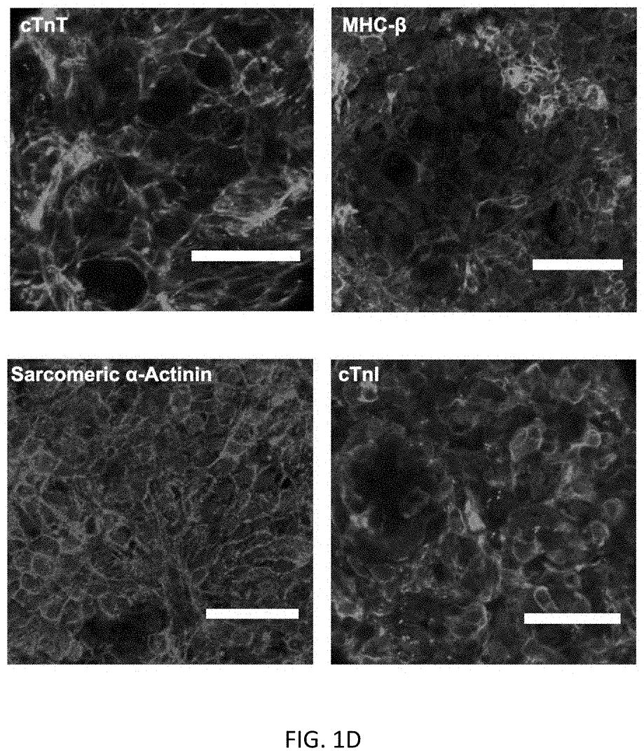

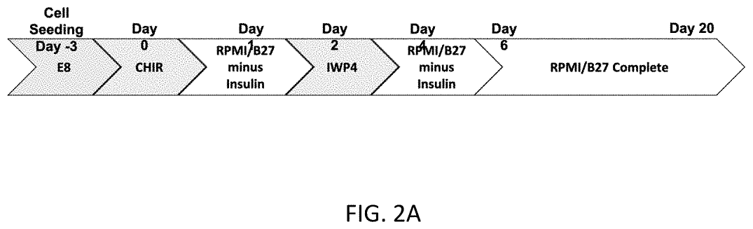

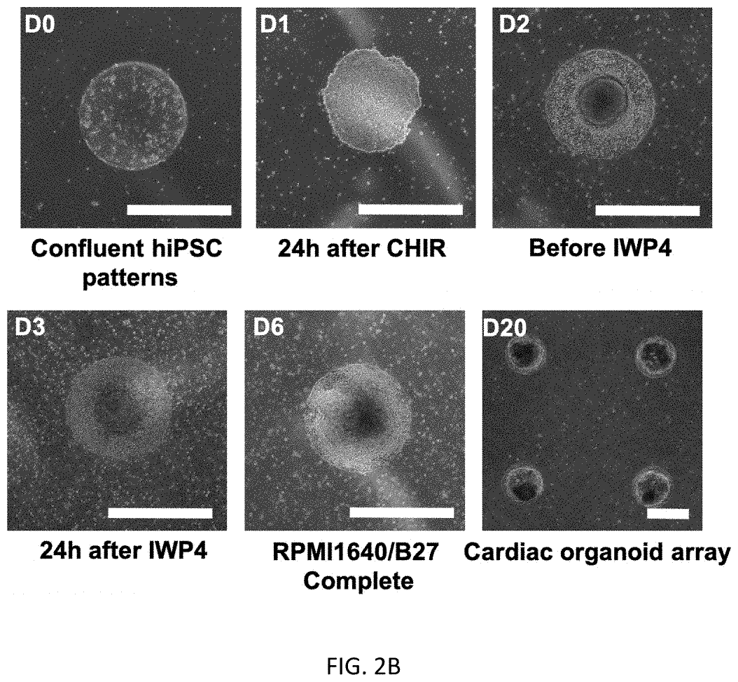

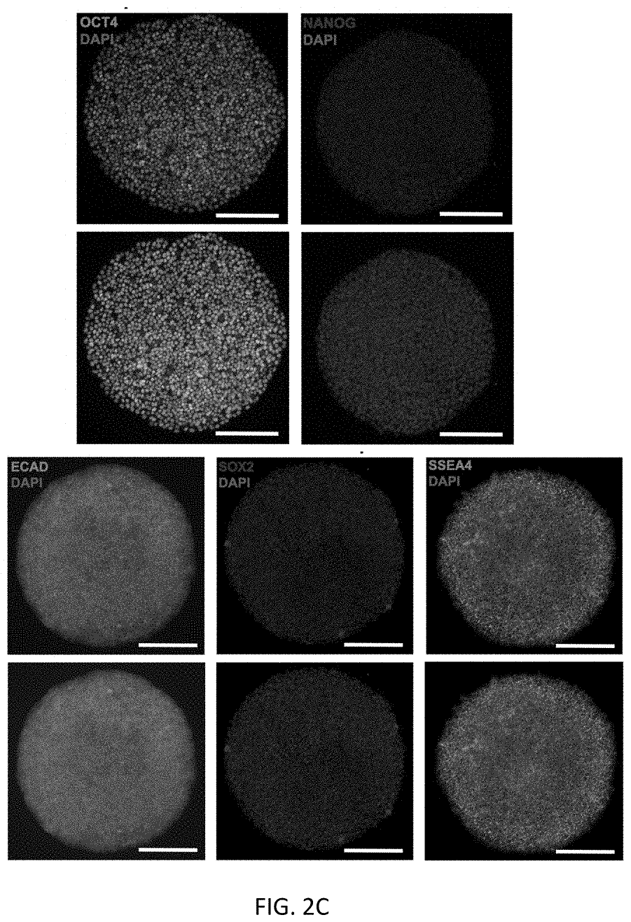

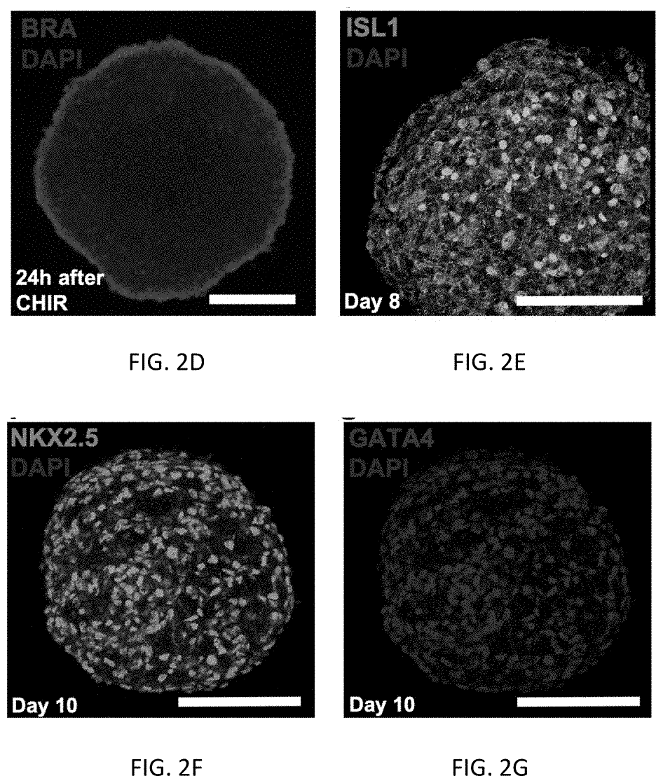

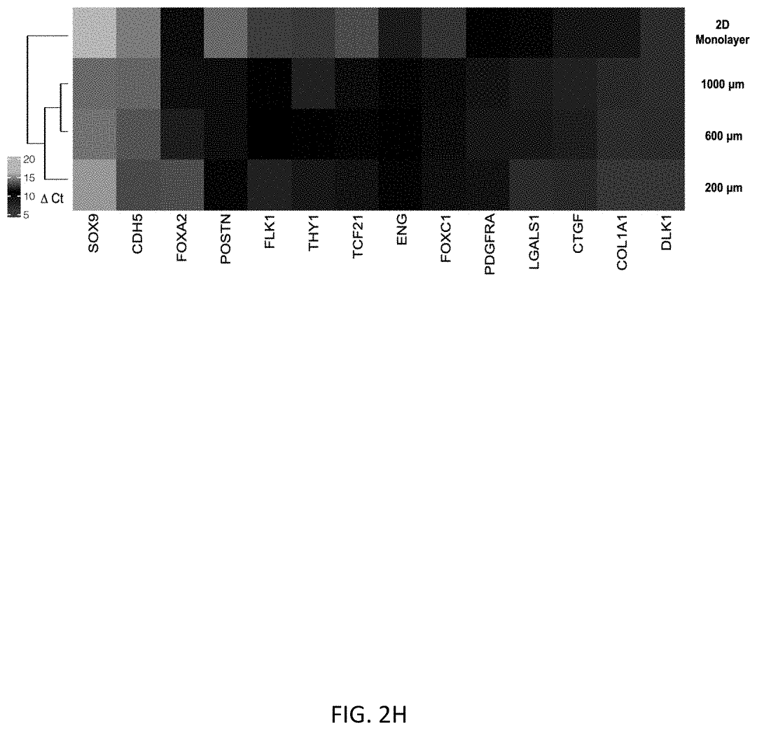

Referring to FIG. 1, wherein like numeral refer to like parts throughout, cardiac differentiation of patterned hiPSCs was used to produce spatially organized cardiac organoids. Geometric confinement to hiPSCs was provided by a poly(ethylene glycol) (PEG)-based micropatterned substrate created by oxygen plasma etching, as seen in FIG. 1(a). hiPSCs seeded on the micropatterned substrates, as seen in FIG. 1(a). Upon confluency, hiPSCs were differentiated via small molecule modulation of the Wnt/β-catenin pathway as seen in FIGS. 2(a) and (b). Patterned hiPSCs were able to proliferate, conform to the pattern geometry and retain pluripotency, as indicated by positive immunofluorescence of OCT4, NANOG, E-cadherin, SOX2 and SSEA4 as seen in FIG. 2(c). This approach generated robust contracting cardiac organoids arrays of 50-100 organoids within 20 days.

At early differentiation stages, the positive expression of mesoderm marker BRA (Day 1) as seen in FIG. 2(d), and cardiac progenitor markers ISL1 (Day 8), NKX2.5 and GATA4 (Day 10) as seen in FIG. 2(e-g) was verified. Expression of early cardiac progenitor makers was distributed across the entire pattern before the cardiac organoids began contracting around Day 12. As the contraction became robust over time, the cardiac tissue compacted towards the center, revealing the underneath stromal cells. At Day 20 of differentiation, cardiomyocytes were primarily differentiated on the center top of the organoids, demonstrated by multiple cardiac-specific markers (FIG. 1c, d): cardiac troponin T, myosin heavy chain β, sarcomeric α-actinin and cardiac troponin I, while smooth muscle-like stromal cells along the pattern perimeter, indicated by positive expression of α-smooth muscle actin, calponin, α-SM22 and vimentin.

The gene expression profile between cardiac organoids from three different pattern sizes (200 μm, 600 μm and 1000 μm in diameter) and traditional 2D monolayer differentiation was compared. In general, gene expression showed upregulation of cardiac-specific genes and downregulation of WNT signaling at Day 20 differentiation, though the gene expression profile had closer similarity amongst different organoids than 2D differentiation (FIG. 1e). The organoids also showed significantly higher gene expression related to TGFβ signaling (TGFB1, TGFB2, TGFB3, TGFBR1, TGFBR1). This might relate to a high content of stromal cells in the organoids under geometric confinement on the patterned substrate as seen in FIG. 2(h). When comparing organoids generated from different pattern sizes, organoids of 600 and 1000 μm showed upregulation of cardiac-specific genes (MYL4, MYH7, NKX2.5), while smaller 200 μm organoids had higher expression of stromal cell genes (ACTA2, TAGLN, VIM). These results illustrated that high geometric confinement from smaller patterns promoted the differentiation into supportive cell types.

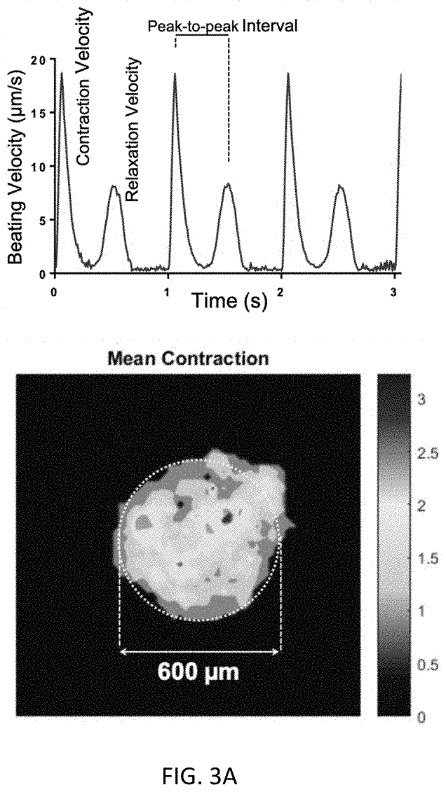

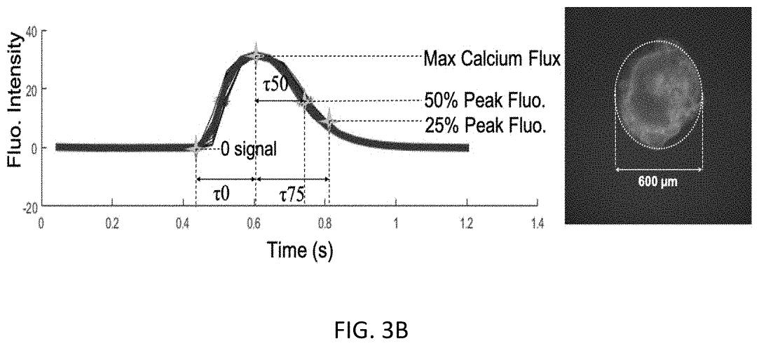

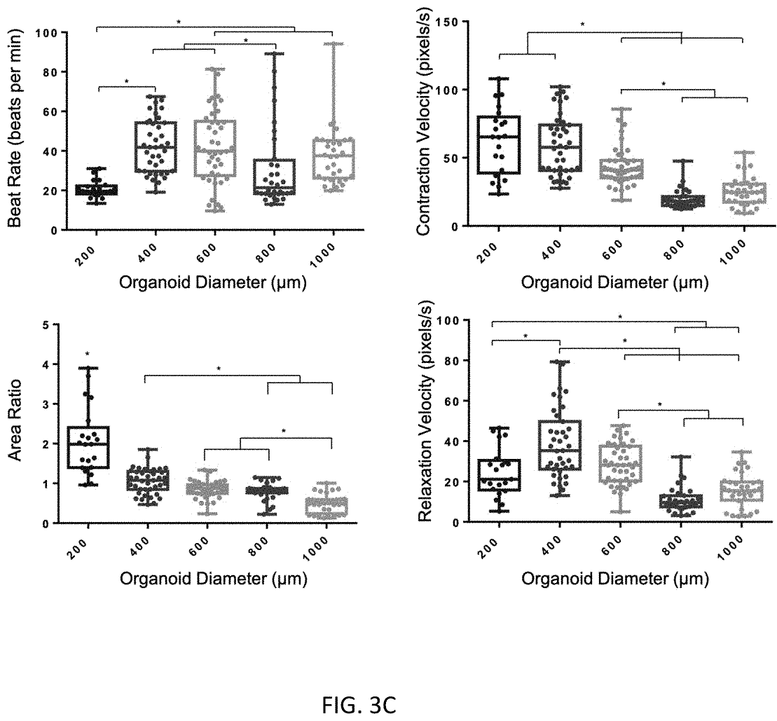

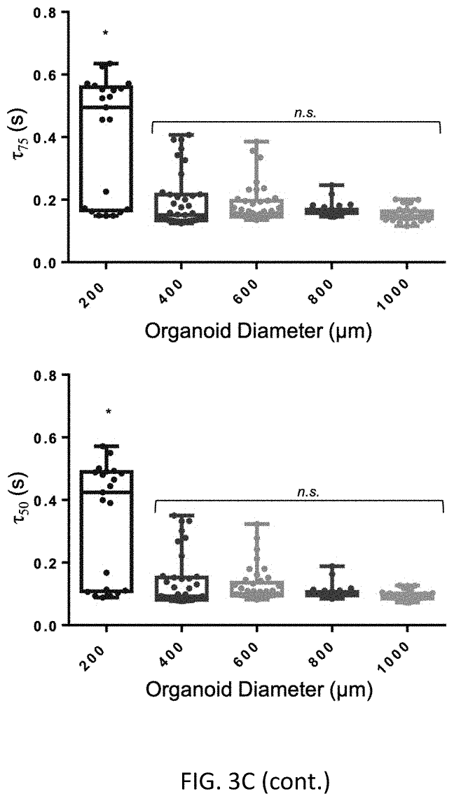

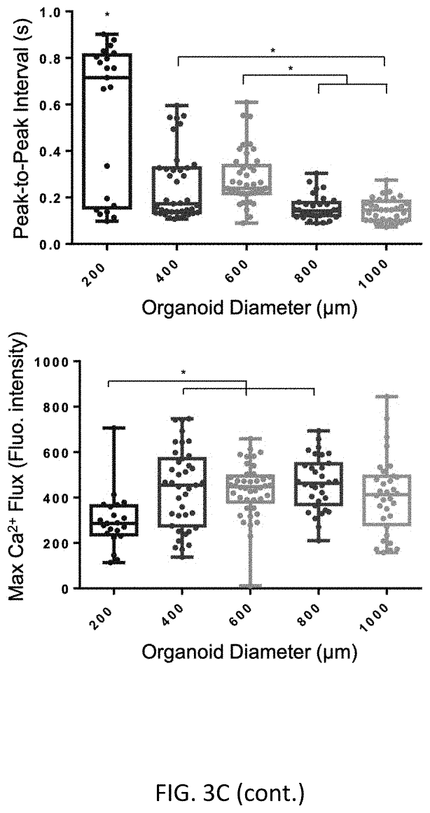

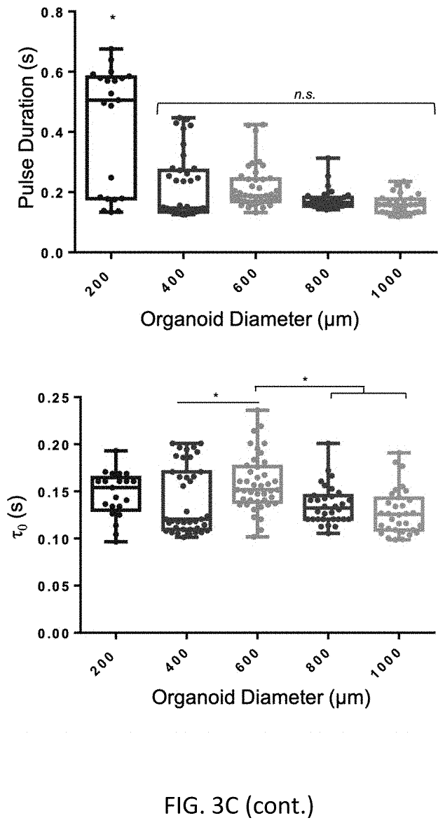

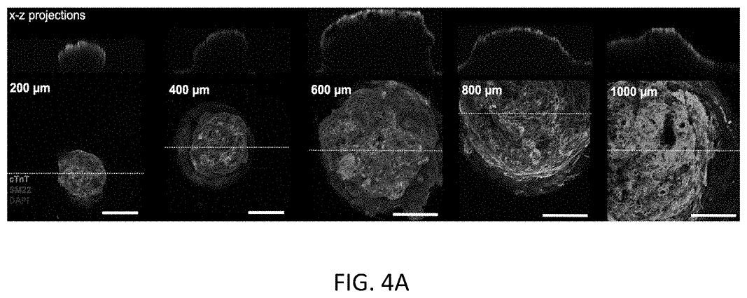

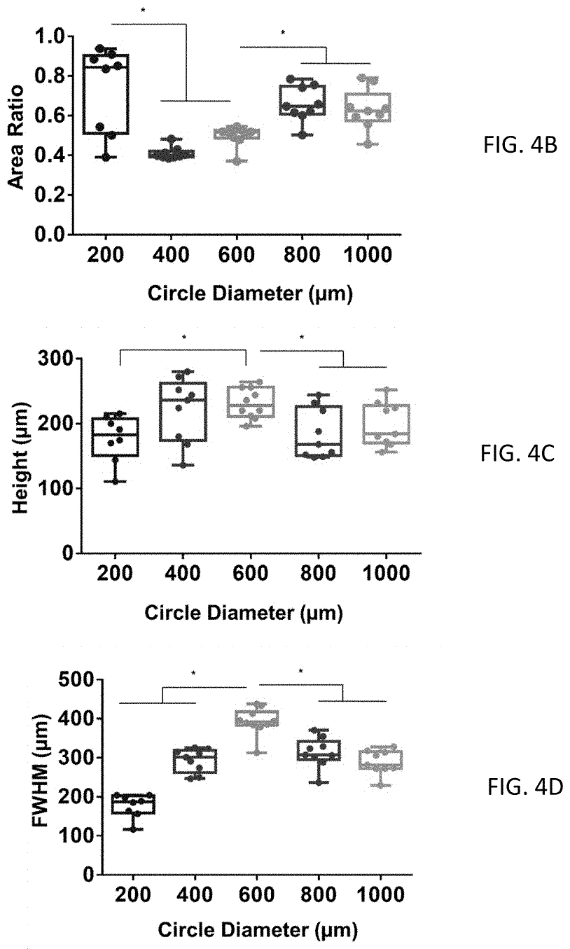

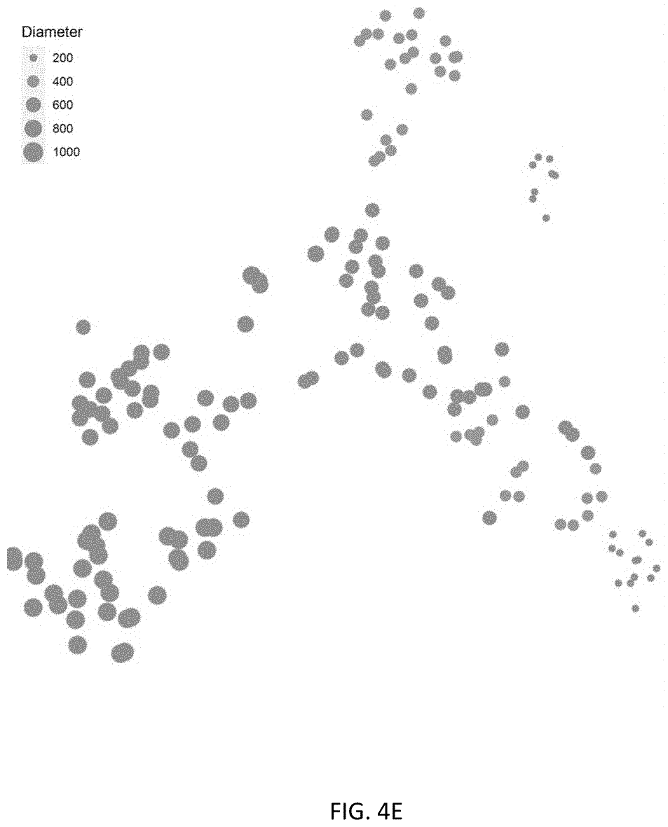

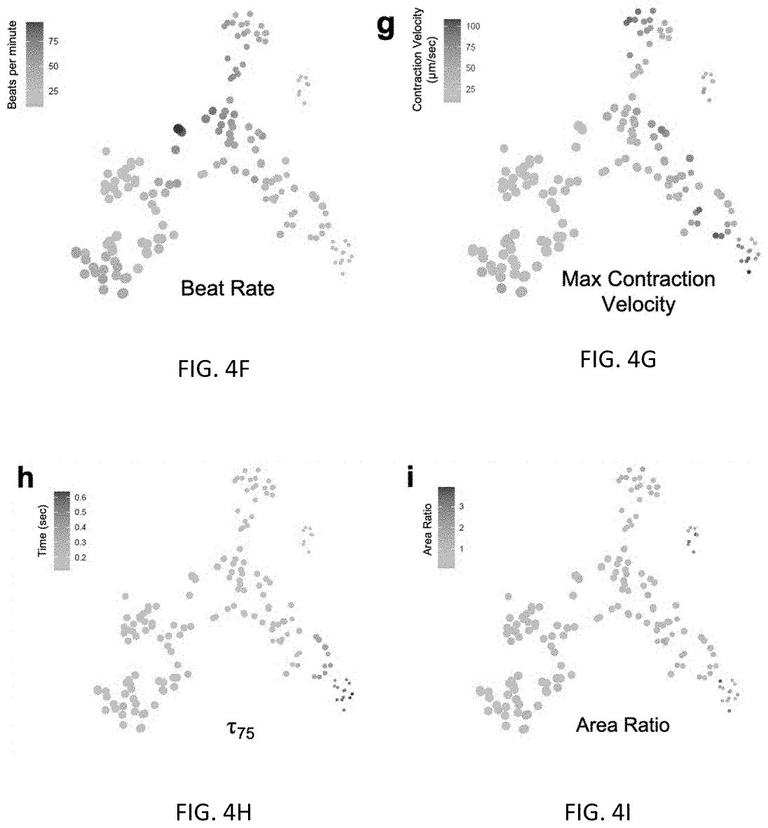

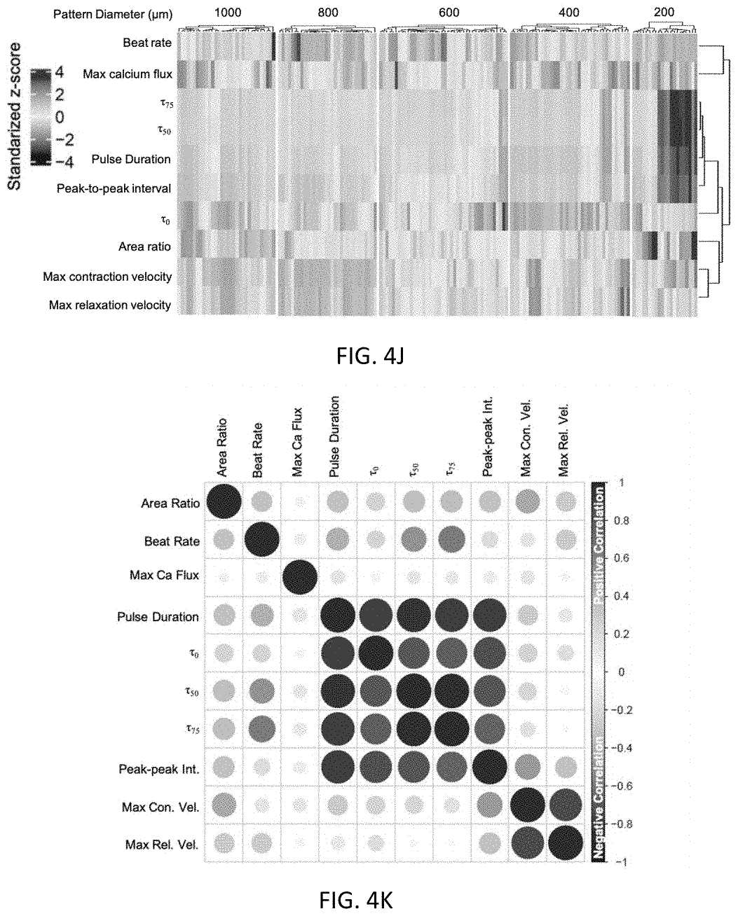

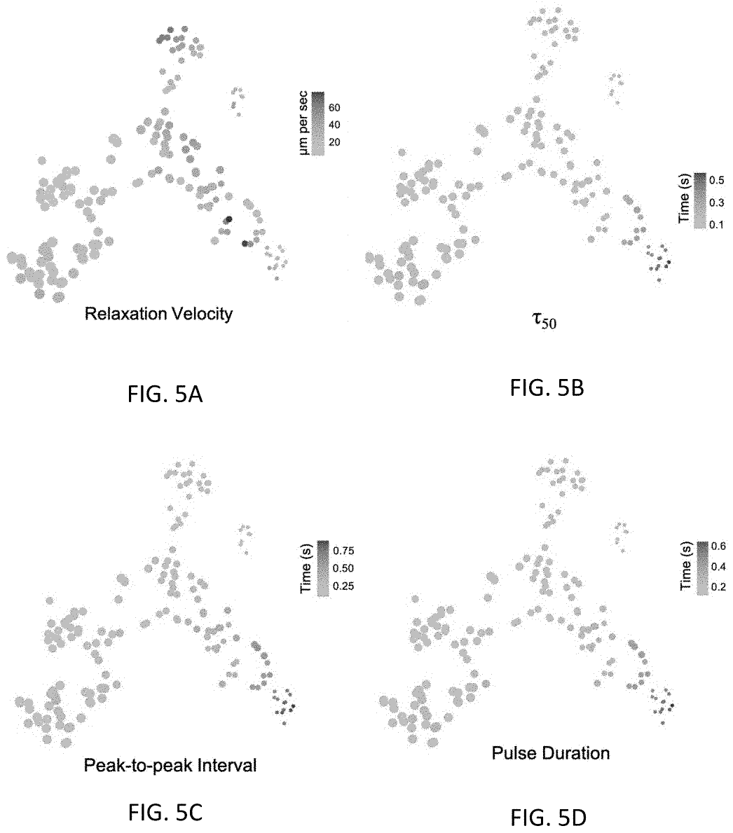

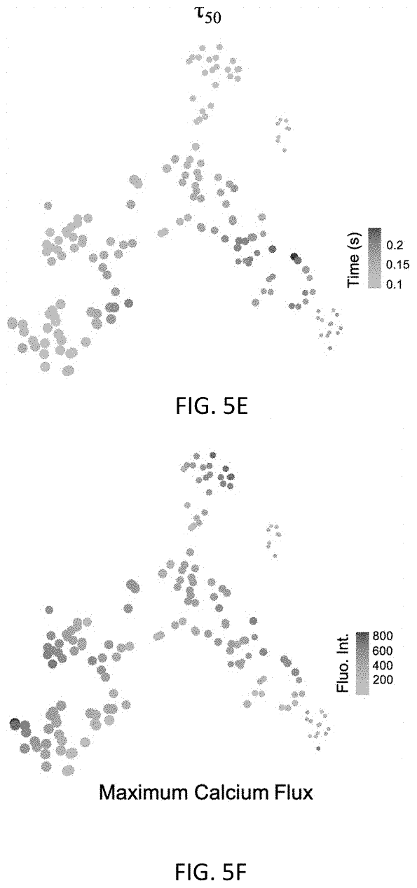

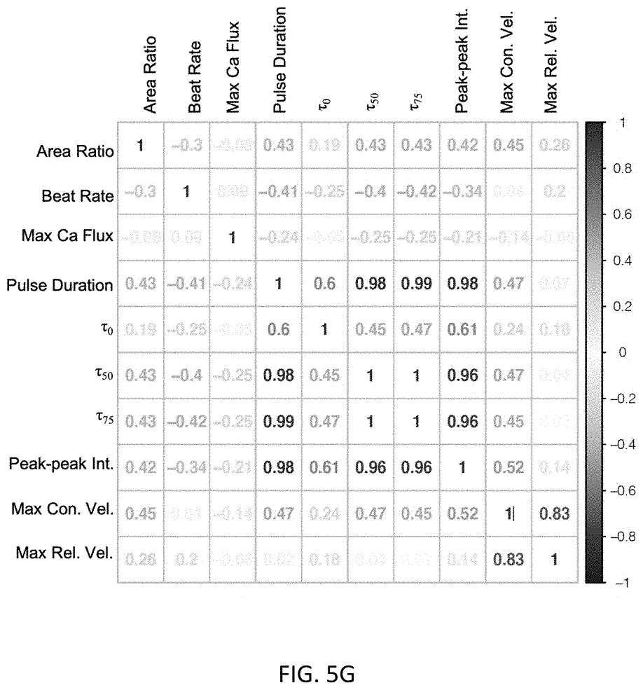

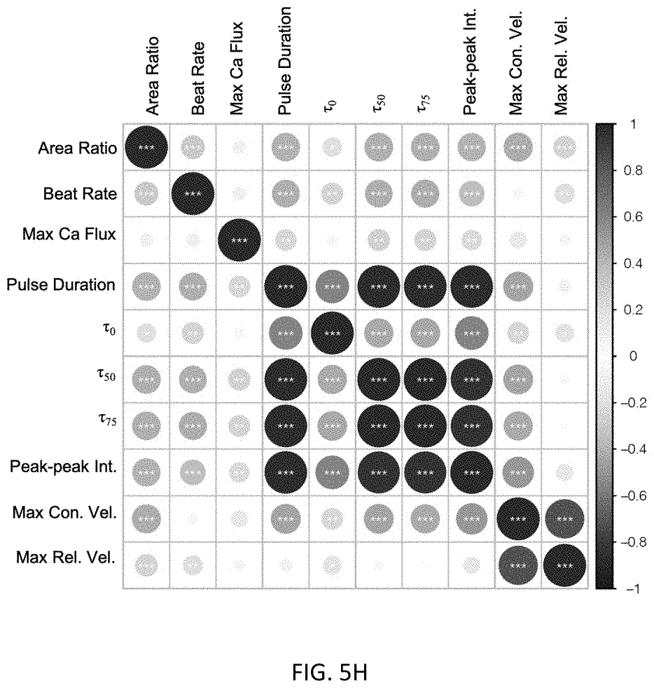

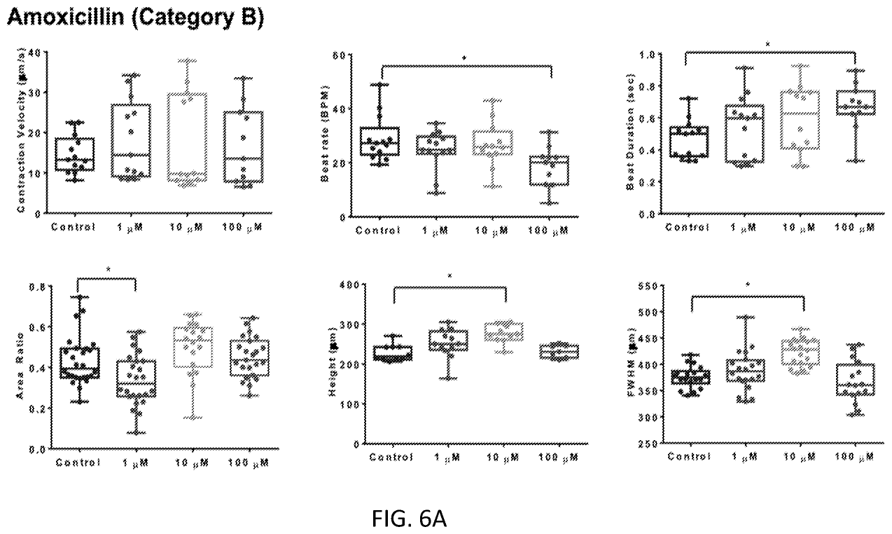

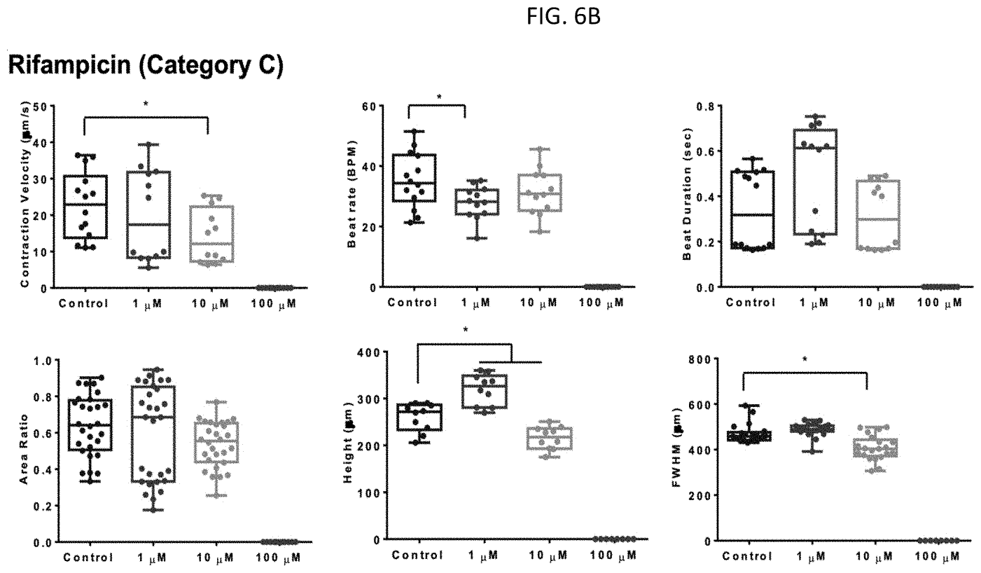

The pattern geometry was found to dictate the structural morphology and contractile physiology of cardiac organoids. Using immunofluorescence staining, the cardiac tissue distribution was measured by calculating the area ratio between the areas of cardiomyocyte differentiation and entire pattern area as seen in FIG. 2(a, b). Using confocal microscopy and 3D image reconstruction, we assessed the three-dimensionality of cardiac organoids by measuring the height and full-width at half-maximum (FWHM) as seen in FIG. 2(c). To study the contractile functions of cardiac organoids, we generated the cardiac organoids from genome-edited hiPSC line with GCaMP6f reporter, which provided the capability to assess the cardiac contractions using brightfield motion tracking analysis as seen in FIG. 3(d). Motion data was integrated with fluorescent calcium flux as seen in FIG. 5a, b) for a comprehensive characterization of contraction functions. t-SNE plots (FIG. 4e-i), together with a heatmap (FIG. 4(k) were generated using quantified contractile function parameters from individual organoids with different pattern sizes: area ratio between GCaMP6 fluorescence and pattern area, beat rate, maximum calcium flux, pulse duration of calcium flux cycle (τ₀, τ₅₀, τ₇₅), peak-to-peak interval, maximum contraction velocities and relaxation velocities as seen in FIG. 5(c).

To examine the effects of pattern size on cardiac organoid development, organoids were generated from circular patterns ranging 200-1000 μm in diameter (FIG. 2a, FIG. 5. The 200 μm patterns did not reliably produce the organoids with a relatively low organoid production efficiency (˜20%), in comparison to all other sizes (˜80%). Albeit with the largest variability, 200 μm circle patterns produced the cardiac organoids with the largest area ratio of cardiac muscles amongst all the sizes (FIG. 4b). Regarding large sizes (800 μm and 1000 μm in diameter), these patterns produced the organoids with a greater area ratio, but lower organoid height than the ones from 400 μm and 600 μm circles (FIG. 4c). Additionally, the FWHM steadily increased with increasing pattern size, peaked at 600 μm patterns and decreased at larger sizes (FIG. 4d). This indicated that 600 μm patterns produced the cardiac organoids with high consistency and large 3D morphology.

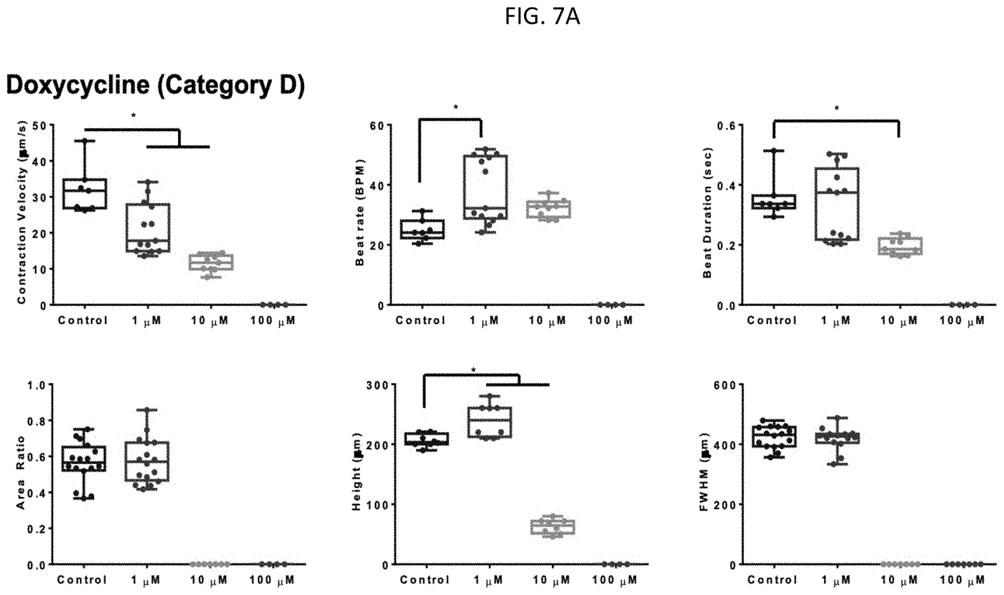

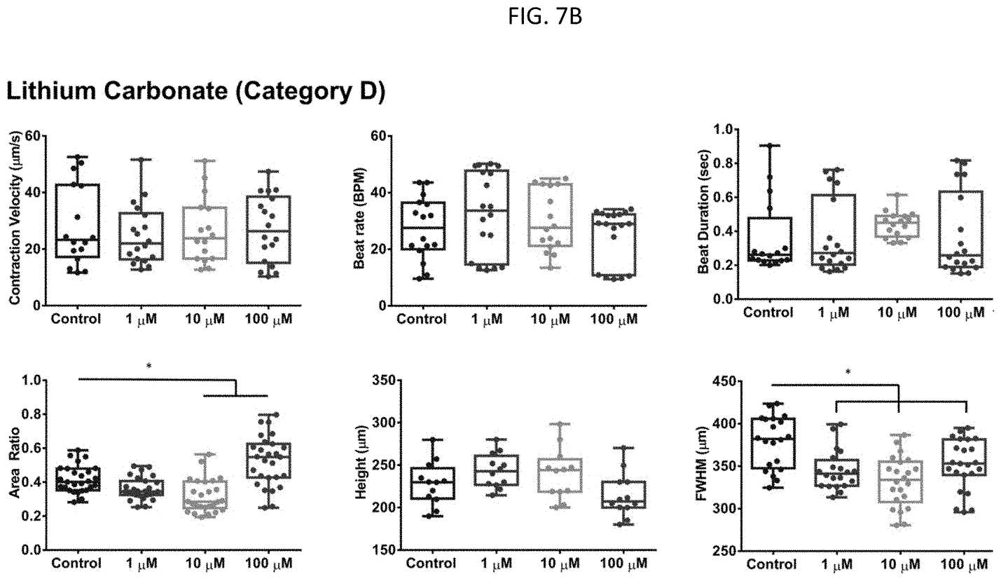

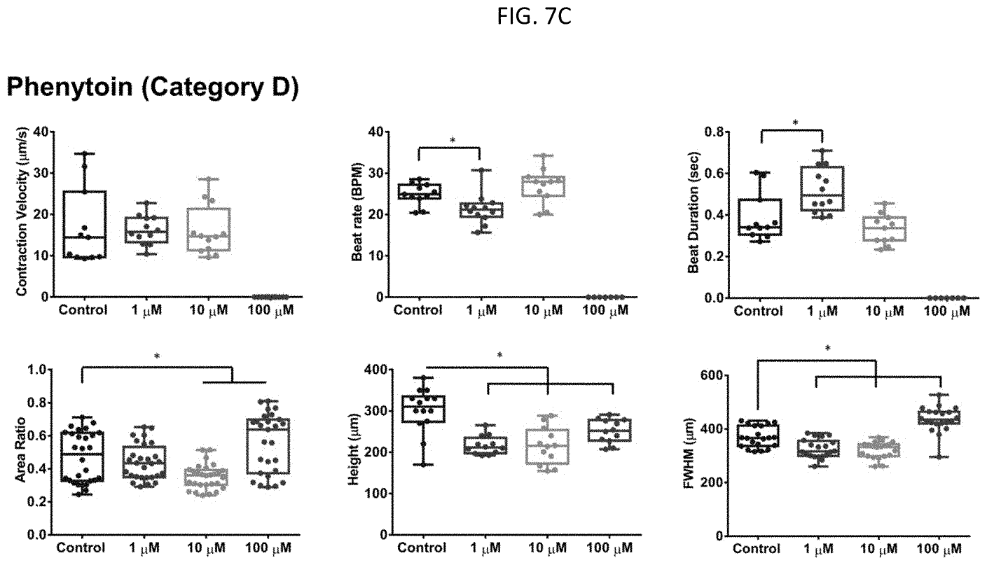

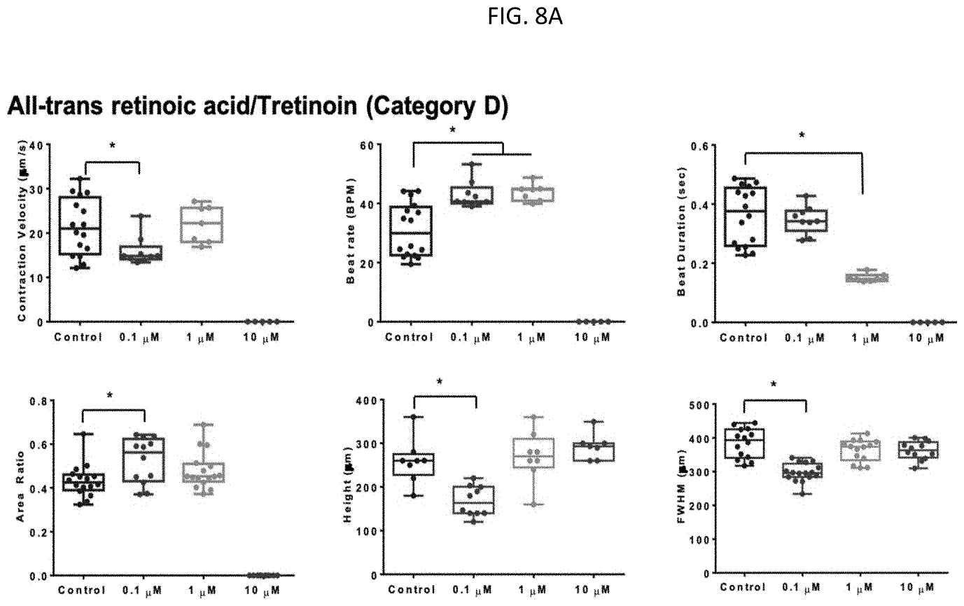

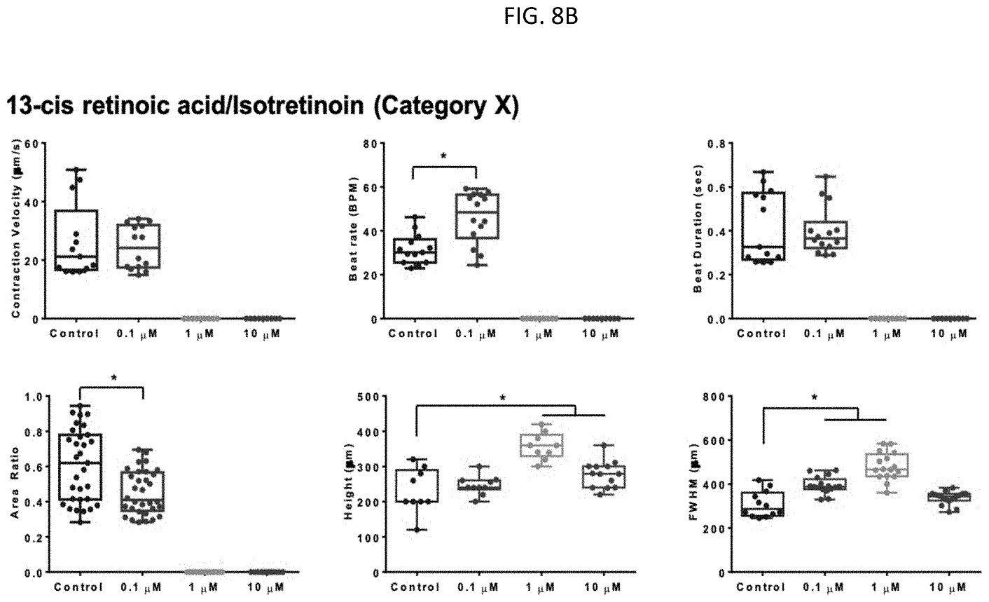

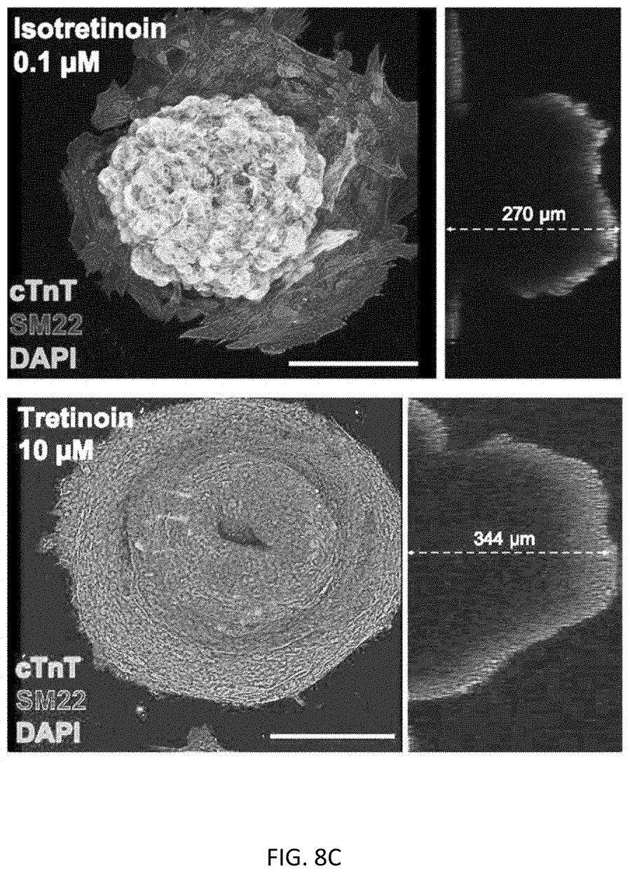

From the t-SNE plot (FIG. 4e) generated from data mining of contraction function parameters as seen in FIG. 7, organoids of larger sizes (600 μm, 800 μm and 1000 μm) were better clustered with high consistency. Organoids from 200 μm and 400 μm patterns showed significant organoid divergence with separated clusters, indicating less consistency in organoid properties. t-SNE plots were used to illustrate the gradients of each parameter across the organoid sample distribution (FIG. 4f-l, FIG. 8(a-f). Organoids of larger patterns exhibited higher beat rate (FIG. 4f), while smaller patterns exhibited higher contraction and relaxation velocities (FIG. 4g, FIG. 8(a). Consistent with the confocal structural analysis, the 200 μm organoids produced the largest area ratio of cardiac tissue relative to pattern size (FIG. 4h). In these t-SNE plots, the divergence of small organoids from 200 μm and 400 μm pattern primarily resulted from the significant differences on parameters of contraction duration (peak-to-peak intervals, pulse duration, τ₅₀, τ₇₅) (FIG. 4i and FIG. 8(b-d). The metrices of τ₀ and max calcium flux exhibited no apparent trends amongst different organoid sizes, as seen in FIG. 8(e, f).

These parameters for the cardiac organoids of different sizes were then compared in the heat map (FIG. 4j). Organoids larger than 200 μm exhibited comparable trends of beat rate and maximum calcium flux, indicating efficient calcium handling and consistent beat frequency. Meanwhile, the contraction duration parameters of peak-to-peak intervals, pulse duration, τ₅₀, and τ₇₅ showed high level of size dependency. The prolongation of contraction duration, especially regarding calcium decay in the cardiac organoids from small patterns, is prone to arrhythmia related to the abnormal diastolic functions. From the correlation matrix (FIG. 4k), strongest correlations were observed between parameters of calcium analysis (τ₀, τ₅₀, τ₇₅, pulse duration), which had negative correlation with beat rate, as seen in FIG. 8(g, h). Furthermore, we were able to establish the correlation across different functional analysis. There was a strong positive correlation between calcium decay from calcium flux analysis and peak-to-peak interval from contractile motion analysis. More importantly, area ratio from morphological analysis is positively correlated with calcium decay and contractile motion velocities from different functional analysis, indicating a structure-function relationship between the relative tissue size and cardiac contraction cycles.

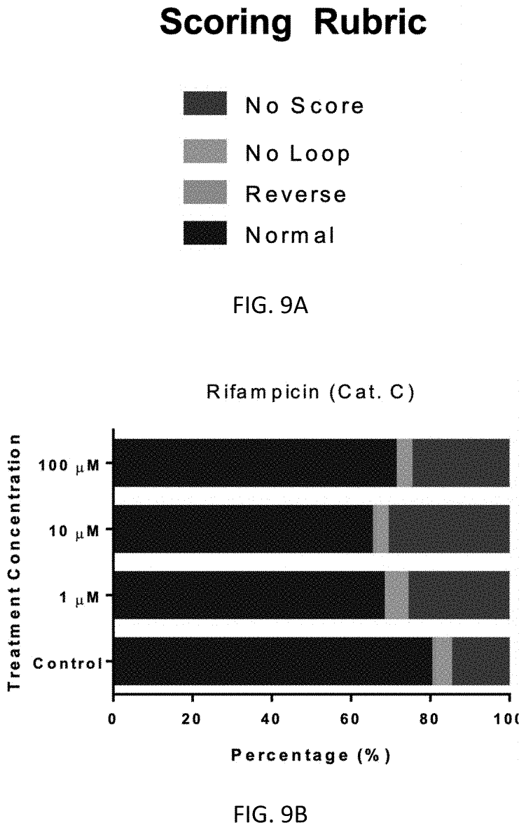

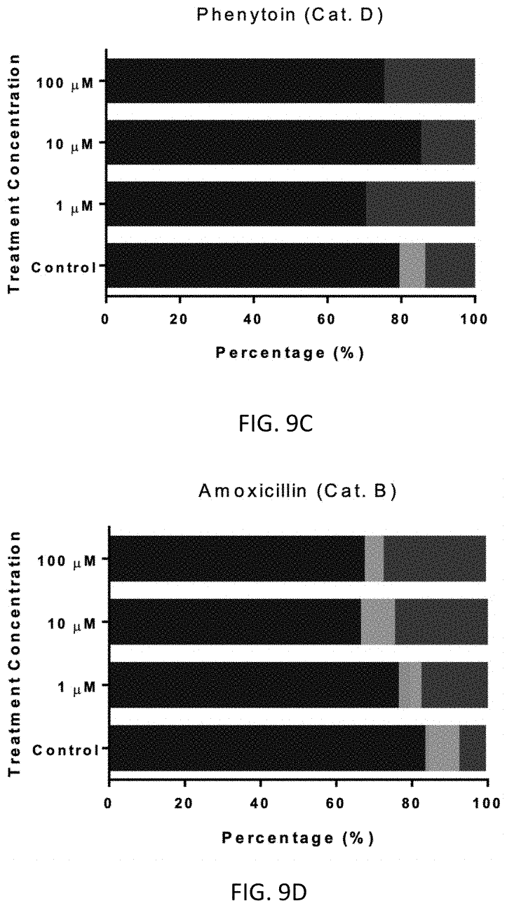

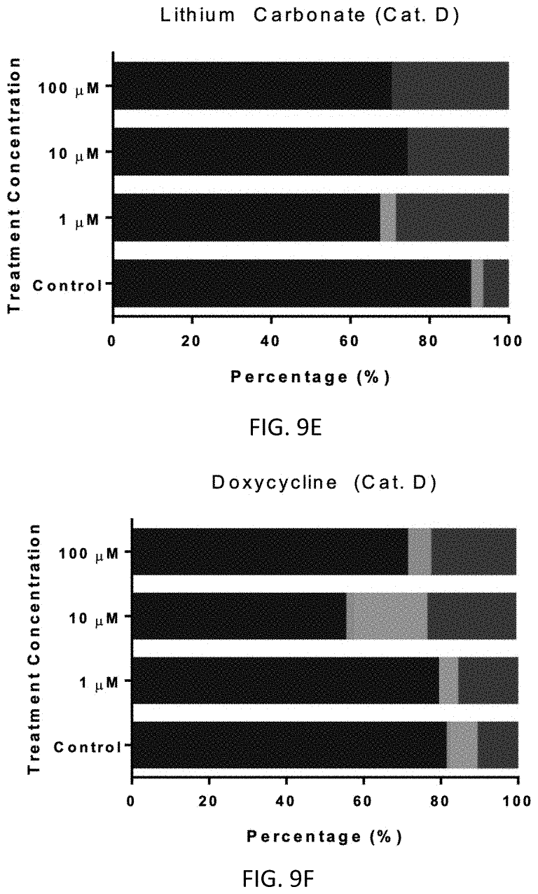

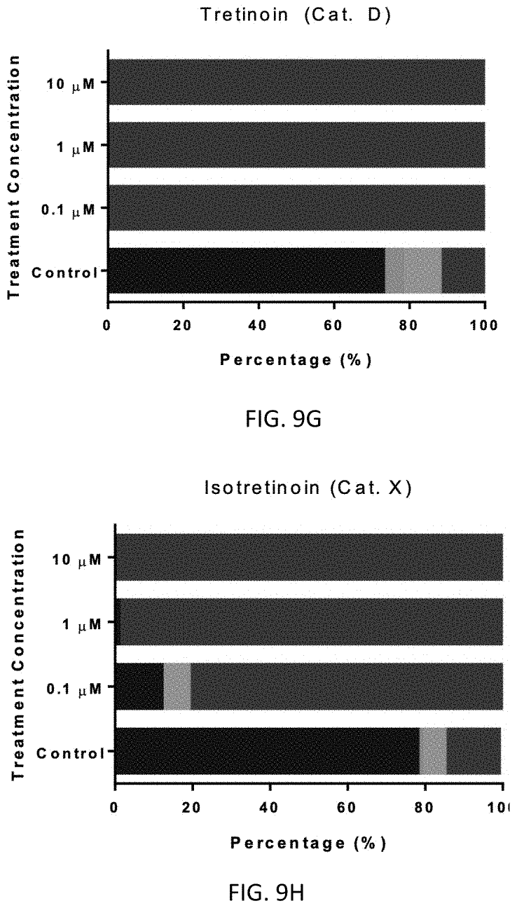

Circle, square, and triangle organoids with the same area as seen in FIG. 9(a) were compared. Cardiac organoids from circular patterns exhibited significantly higher contraction velocity, lower beat rate and longer beat duration as seen in FIG. 9(b-d). Furthermore, circle patterns produced the organoids with larger area ratio, height and FWHM than squares and triangles as seen in FIG. 9(e-g). Overall, the circular pattern geometry promoted more robust formation of cardiac organoids with large size and high contractile functions. These observations illustrated that the development of cardiac organoids was affected by pattern shape, in which the angular geometries presented high physical constraint to the cells and promoted a higher degree of differentiation into the stromal cell types, rather than cardiomyocytes. By plotting the structure-function correlations and structure-structure, a decline in the beat rate that corresponded to the increasing area ratio as seen in FIG. 9(h, i) was observed as a negative correlation between the cardiac tissue size and the beat rate.

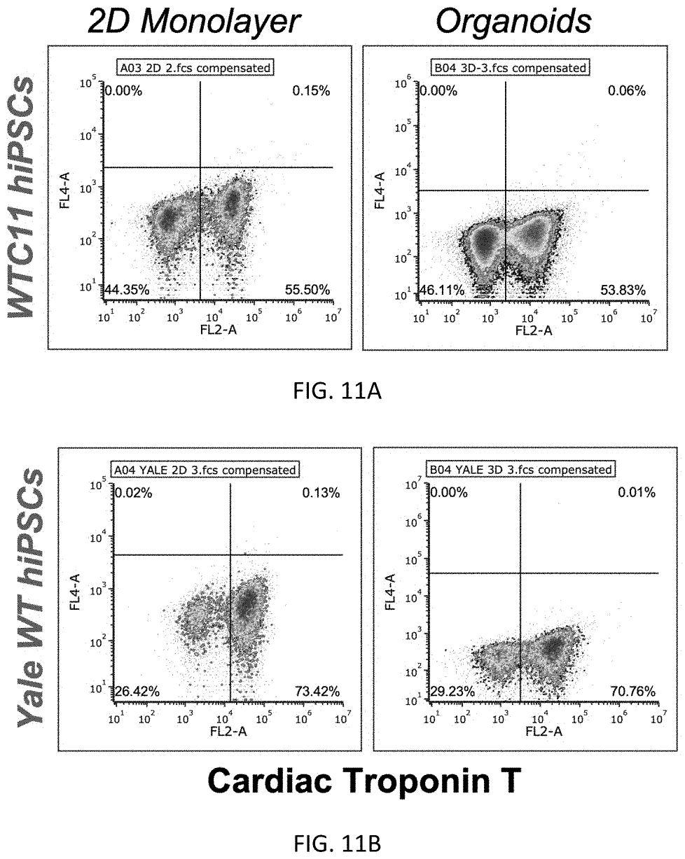

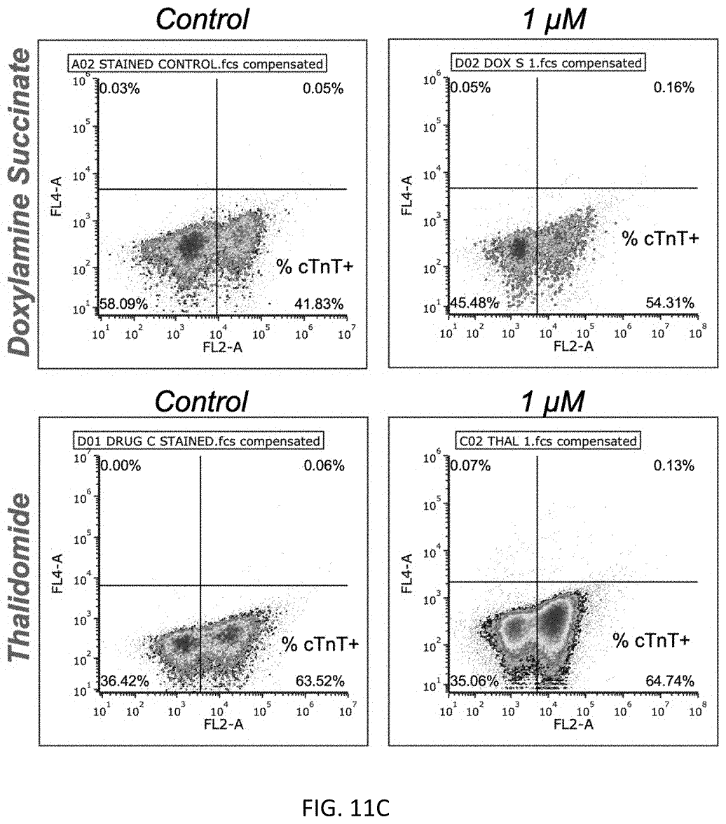

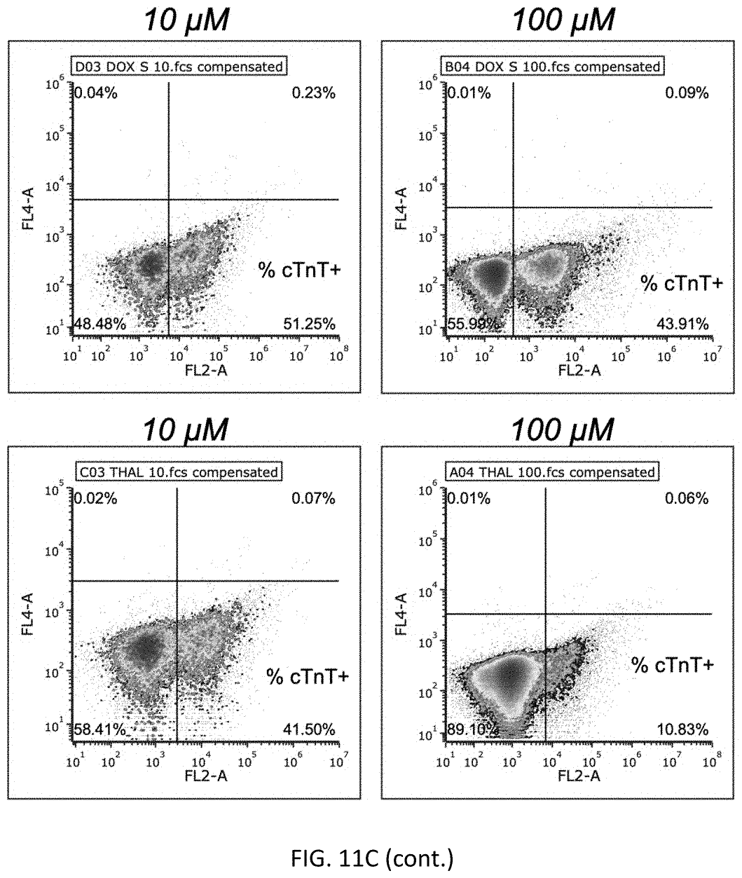

Cardiac organoids according to the present invention can be used for an in vitro assay for cardiac developmental toxicity. The heart is the first functional organ to form, thus cardiac differentiation is often used as a key evaluation for developmental toxicity. Since 600 μm-diameter circular patterns gave robust organoid production, large 3D morphology, consistent contractile functions and high level of cardiac-specific differentiation, arrays of cardiac organoids from this geometry were used to test the capabilities of this platform to be implemented as a cardiac developmental toxicity assay. Using flow cytometry, we quantified the consistency of cardiac differentiation efficiency of organoids and 2D monolayer differentiation from 5 different batches (n=5) across 10 passages of hiPSCs from two different lines. Both organoids of 600 μm and 2D differentiation produced over 50% cells that were positive for cardiac troponin T as seen in FIG. 11(a, b). These results illustrated that cardiac organoids can be generated consistently across multiple cell batches and multiple hiPSC lines. Furthermore, the result of ˜50% cTnT+ cells from flow cytometry was consistent with the area ratio calculation, where 600-μm organoids exhibited an average area ratio of approximately 0.5 (FIG. 4b). Since the percentage of cTnT+ cells that composed organoids was comparable to the area ratio metric, it may be inferred that the area ratio is an appropriate metric to approximate cardiac differentiation efficiency for drug screening purposes.

Nine drugs, as seen in Table 1 below, used in connection with the present invention covered the entire spectrum of the past FDA pregnancy category system that ranked drugs from A (safe) to X (toxic) based on predicted teratogenic risk.

TABLE 1

| | | Pregnancy | Concentrations |
|---|---|---|---|
| Drug | Medicinal Use | Category | Tested |
| Doxylamine | Antihistamine, | A | 1 μM, 10 μM, 100 μM |
| Succinate | sleeping aid | | |
| Amoxicillin | Antibiotic | B | 1 μM, 10 μM, 100 μM |
| | (strep, middle ear | | |
| | infections etc.) | | |
| Rifampicin | Antibiotic (TB, | C | 1 μM, 10 μM, 100 μM |
| | leprosy etc.) | | |
| Lithium | Bipolar | D | 1 μM, 10 μM, 100 μM |
| Carbonate | antidepressant | | |
| Phenytoin | Anticonvulsant | D | 1 μM, 10 μM, 100 μM |
| Doxycycline | Antibiotic | D | 1 μM, 10 μM, 100 μM |
| All-trans-RA | Acne treatment | D | 0.1 μM, 1 μM, 10 μM |
| (Tretinoin) | | | |
| 13-cis-RA | Acne treatment | X | 0.1 μM, 1 μM, 10 μM |
| (Isotretinoin) | | | |
| Thalidomide | Myeloma, | X | 1 μM, 10 μM, 100 μM |
| | Inflammation | | |

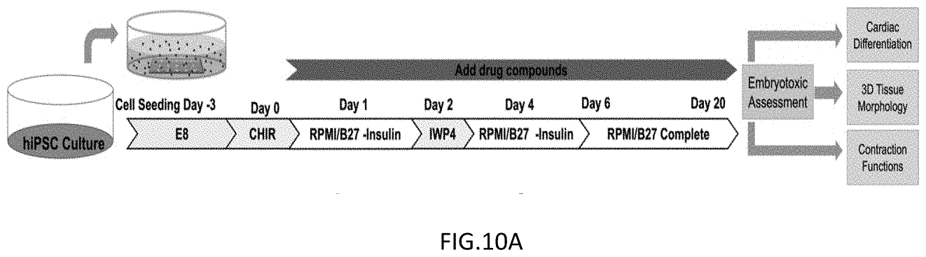

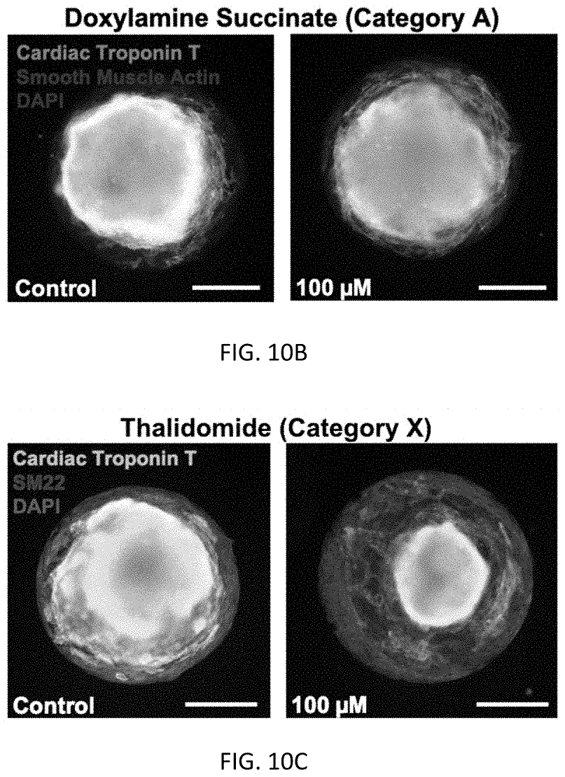

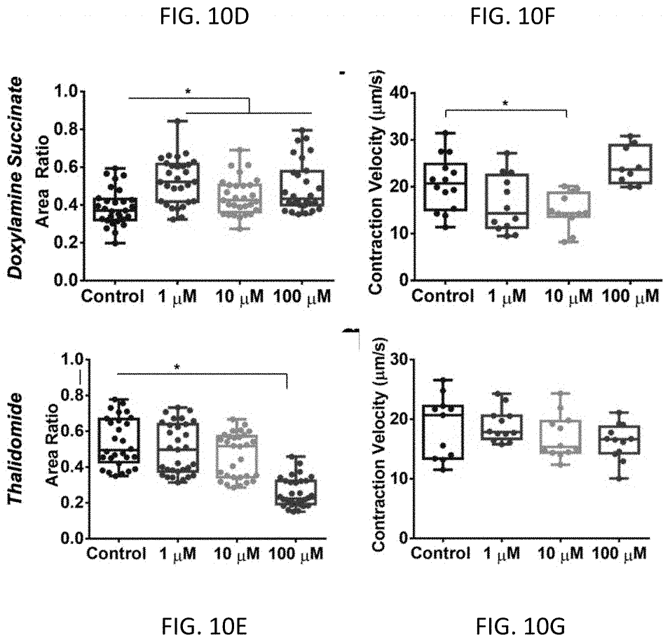

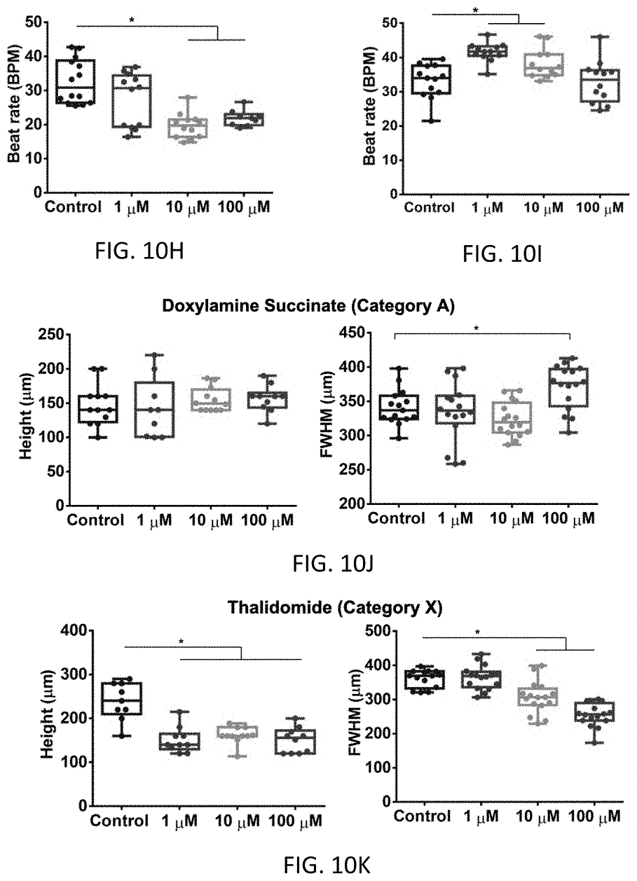

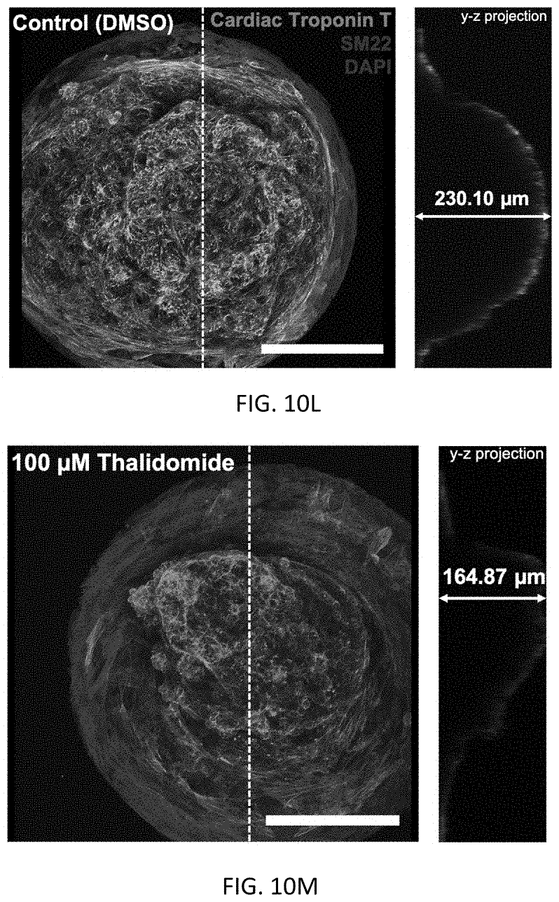

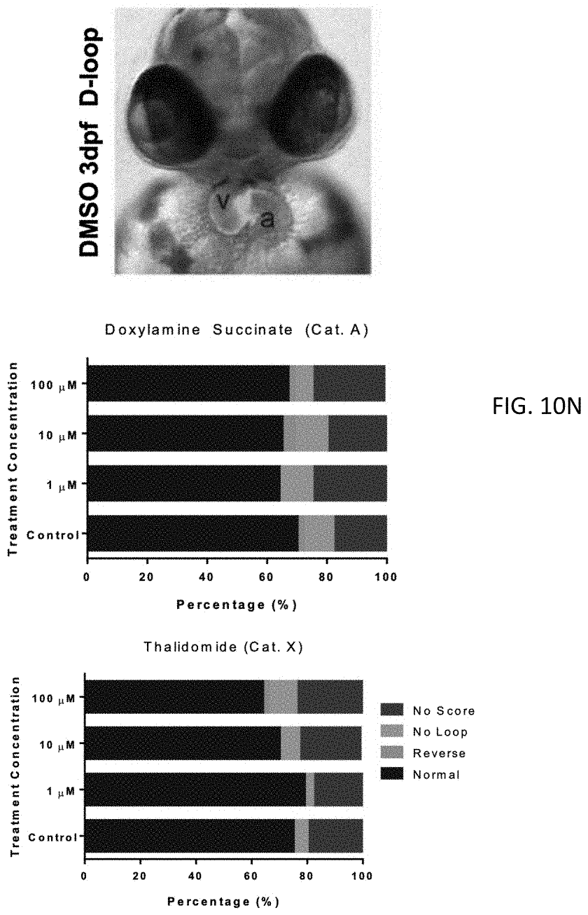

The drugs were individually introduced on Day 1 of differentiation, 24 hours after treatment of GSK3 inhibitor CHIR99021 (FIG. 10a). This initial timepoint was chosen because it specifically targets cardiac differentiation after mesoderm induction. First, two drugs between category A (doxylamine succinate) and category X (thalidomide) were compared. Doxylamine succinate (Category A, FIG. 10b) treatment had no negative effects on cardiac differentiation, whereas thalidomide (Category X, FIG. 10c) produced the abnormal cardiac organoids with less cardiac tissue coverage relative to the untreated controls (FIG. 10(d, e)). A similar trend was seen when quantifying cardiac differentiation efficiency via flow cytometry analysis as seen in FIG. 11(c). Doxylamine succinate had prominent effects on the contractile functions with lower contraction velocity and slower beat rate (FIG. 10f, h) due to its anticholinergic effects as an H1 receptor antagonist. Generally used as a sleeping aid due to its relaxant effects, doxylamine succinate is also prescribed as an analgesic to reduce muscle tension. It is possible that the reduction of muscle contraction due to doxylamine treatment led to lessened tissue compaction during organoid development, which resulted in an increase of area ratio. In contrast, there is no significant effect from thalidomide on the contraction velocity (FIG. 10g), though high concentration of drug dosage led to high variability in the beat rate (FIG. 10i). More importantly, high concentration of thalidomide (100 μM) impaired the 3D morphology of the cardiac organoids with significantly lower height and FWHM relative to organoids treated with doxylamine succinate (FIG. 10j) and to the controls (FIG. 10k-m). The impairment of cardiac tissue is potentially caused by FGF antagonism of this drug, resulting in decreased muscle development. These results indicated that exposure of a well-known teratogen resulted in severe impairment to hiPSC differentiation and organization into 3D cardiac organoids, which confirmed that the organoid model of the present invention was sensitive to the morphological defects as a result of drug exposure.

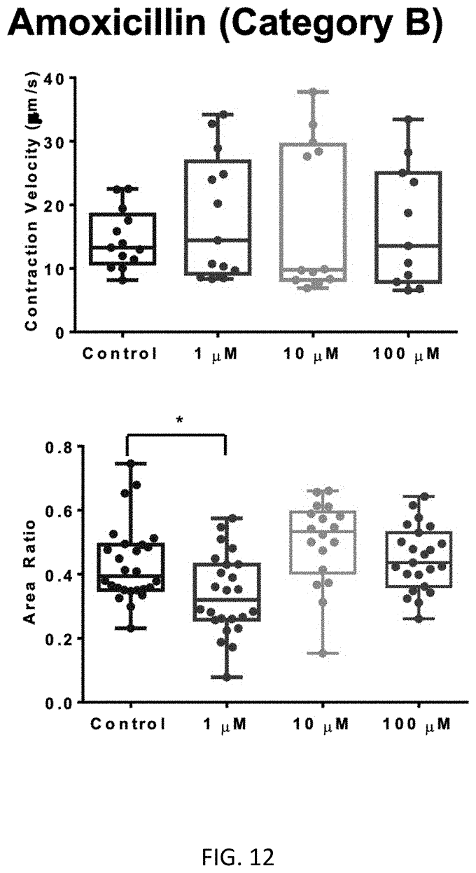

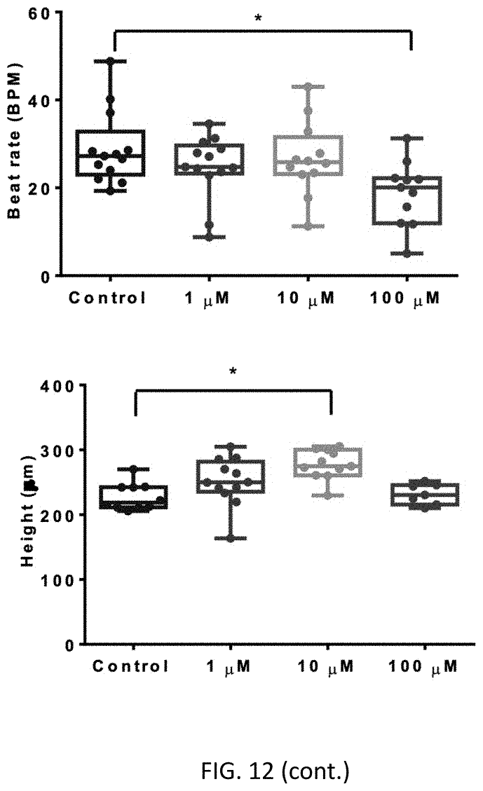

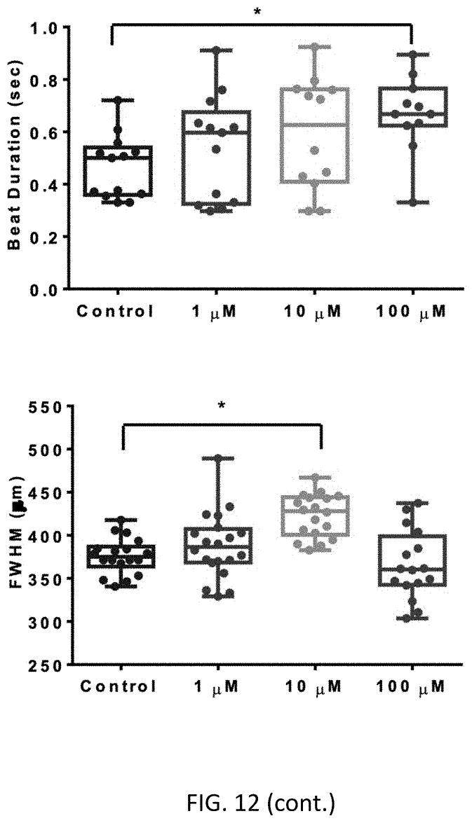

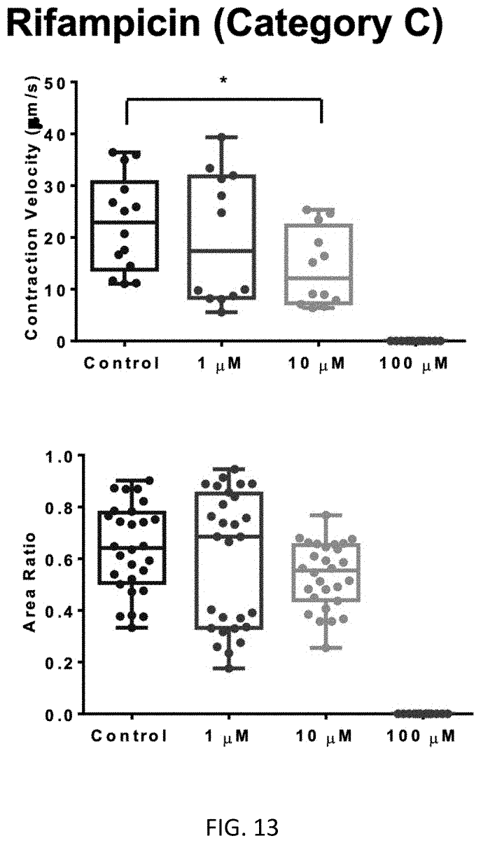

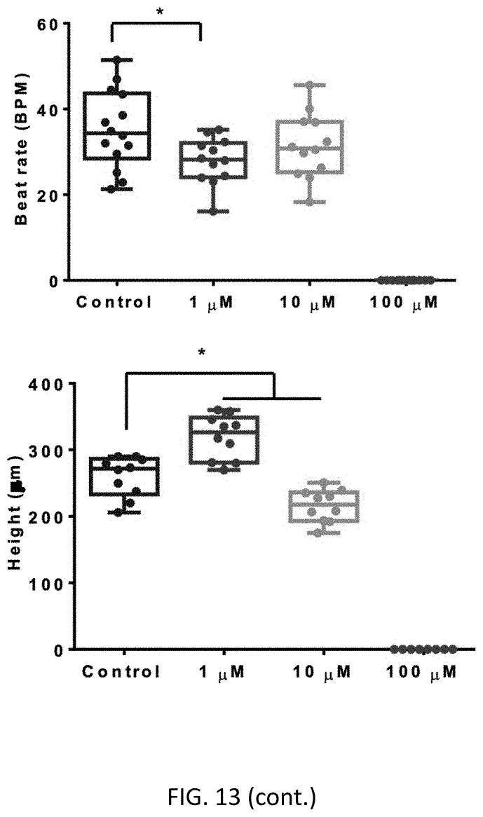

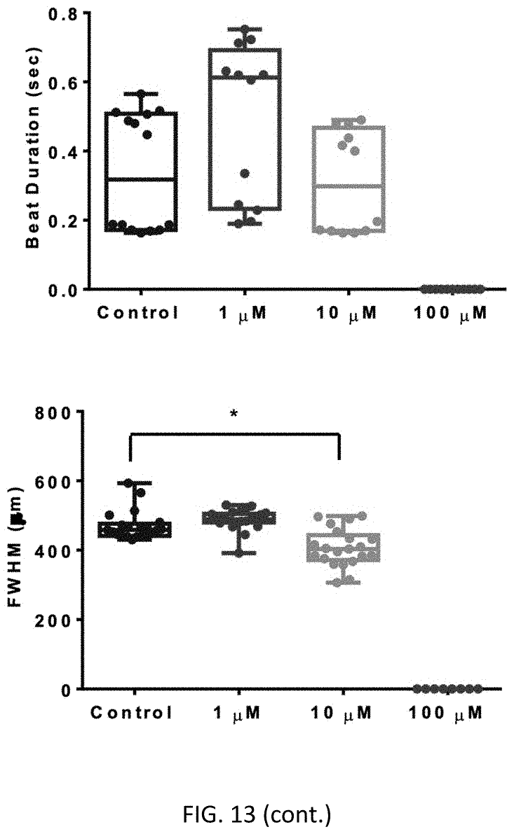

Next, cardiac developmental toxicity of drugs from the other pregnancy categories were tested. Three antibiotic drugs tested on the cardiac organoids showed increased developmental toxicity with the increase of their risk classification in the pregnancy category. Amoxicillin (category B antibiotics) in mammalian cells has been shown to induce DNA lesions as a result of amoxicillin-induced oxidative stress. In the organoids, amoxicillin showed no clear toxic effect on either structure or functions of the cardiac organoids at all three tested concentrations (see FIG. 12). Rifampicin (category C antibiotics) targeting bacterial RNA and DNA for its effectiveness, has been shown to inhibit protein synthesis in mammalian cells. Rifampicin showed severe developmental toxicity at a high concentration (100 μM) with no organoid formation (see FIG. 13). Doxycycline (category D antibiotics) inhibits the synthesis of bacterial proteins by binding to the 30S ribosomal subunit, but also showed adverse effects on mitochondrial ribosomes within mammalian cells. Doxycycline treatment resulted in severe impairment on cardiac differentiation and organoid formation even at a moderate concentration (10 μM) (see FIG. 14a).

Other category D dugs with various therapeutic applications were tested. Lithium carbonate (category D antidepressant), which inhibits the PKC signaling for its psychiatric medication purpose, appeared to inhibit phosphatidylinositol cycle and Wnt pathway activation, based on mEST assays. Exposure of lithium did not affect the contractile functions of the cardiac organoids but exhibited mild toxicity to the organoid formation measured by area ratio and FWHM (see FIG. 14b). Phenytoin (category D anticonvulsant) protects against seizures via voltage-dependent antagonism of voltage-gated sodium channels. Phenytoin exposure produced smaller cardiac organoids than controls, but still maintained structural integrity (see FIG. 14c). Due to its inhibition on sodium channels, the cardiac organoids stopped beating at a high concentration (100 μM), until the drug was removed to allow the organoids to recover the contractile functions. Tretinoin (all-trans-retinoic acid, category D retinoid) treatment completely abolished the cardiac differentiation at the concentration as low as 10 μM, but still produced the organoids with comparable height and overall size as controls (see FIG. 15a).

Overexposure to retinoids was shown to result in birth defects from retinoic acid deficiency with decreased levels of retinoic acid-producing enzymes. Hence, we also tested isotretinoin (13-cis-retinoic acid, category X retinoid) (see FIG. 15b) on the cardiac organoids. Similar to tretinoin, isotretinoin produced large organoids at all tested concentrations, but totally abolished the cardiac differentiation at even lower concentration at 1 μM. This implied that retinoids caused severe impairment to the cardiac differentiation, but not tissue growth. Overall, these results verified that this cardiac organoid model was sensitive to the drug-induced cardiac developmental toxicity and offered the capability of morphological scoring based on the 3D tissue formation, which is often not available from other stem cell-based in vitro assays.

Finally, the developmental toxicity of these drugs between cardiac organoid model and zebrafish whole embryo culture (zWEC), a well-established toxicity assay with promising potential to screen for teratogenicity, was tested. Transgenic Tg(my17:GFP) with GFP only in cardiomyocytes was used, which allowed scoring of myocardial development and heart tube looping at 48 hours post-fertilization (hpf) (FIG. 10n, see FIG. 16). Live zebrafish embryos were collected and exposed to the identical drugs at identical concentrations used in the cardiac organoid assay. Consistent with the organoid model, exposure with doxylamine succinate (Category A) had negligible effect on the zebrafish embryonic heart development, as there was a considerable proportion of embryos exhibiting normal D-looped heart across all concentrations. However, the effects of thalidomide on zebrafish embryonic heart development were not as significant as what was observed in the organoid model. The proportion of normal heart looping did not notably decrease at higher concentrations of thalidomide (FIG. 3n). Rifampicin (see FIG. 16b) and phenytoin (see FIG. 16d) showed mild embryotoxic effects to the zebrafish embryos at the highest concentration, while amoxicillin (Supplemental FIG. 13a), lithium carbonate (see FIG. 16c) and doxycycline (see FIG. 16e) showed moderate toxicity. Retinoic acid derivatives, tretinoin (see FIG. 16f) and isotretinoin (see FIG. 16g), caused severe defects to the embryo heart development. At low concentration (0.1 μM), embryos had severe heart defects, including smaller size and abnormal morphology. At higher concentrations, the hearts failed to develop, as indicated by 0% of embryos expressing the GFP transgene. Upon comparing the organoid model with the zWEC model, developmental toxicity was seen that was comparable for between these two systems for most of drugs (doxylamine succinate, amoxicillin, lithium carbonate, phenytoin, tretinoin, and isotretinoin). However, rifampicin, doxycycline and thalidomide showed distinct mismatch between these two model systems. The results may be due to a number of factors, including species differences, method of drug exposure, and differences in the range of effective treatment concentrations.

The present invention thus allows for the generation of cardiac organoids starting with 2D micropatterned hiPSC colonies, allowing for cell self-organization into 3D tissue structures during the differentiation process under geometric confinement. This approach provides the ability to create cardiac organoids that replicate, to a certain extent, the biological process of tissue self-assembly and morphogenesis during early heart formation. As many commonly reported birth defects are heart related, the potential for generating cardiac defects is a primary concern in determining drug developmental toxicity. The cardiac organoid approach of the present invention allows for the evaluation of human-specific drug-induced developmental toxicity based on the disruption of forming correct 3D organoid structures and developing normal cardiac contractile functions. By exposing the cardiac organoids to a range of drugs with different risks, an overall increase of teratogenic severity on cardiac organoid formation was found, corresponding to an increase of test concentrations, and an increase of risk category from A to X. Especially, category D drugs (phenytoin, lithium, doxycycline and tretinoin) showed diverse effects on developmental toxicity. Surprisingly, exposure of lithium only showed mild developmental toxicity of slight reduction in cardiac differentiation and organoid formation, although there has been a long debate of this drugs' developmental toxicity, especially resulting in congenital heart defects.

For highly toxic drugs, doxycycline failed to create 3D organoids due to massive cell apoptosis at high concentrations, whereas exposure of tretinoin created supersized organoids, but abolished all cardiac differentiation. The drug response results of thalidomide were generally consistent with published works of embryotoxicity using both whole embryo and in vitro stem cell models. In testing of the present invention, retinoids impaired the cardiac differentiation but promoted the formation of giant tissues. It is possible that progenitor cells in the retinoid-treated organoids retained a high proliferative capacity to give rise large tissue growth, but inhibited the terminal differentiation of cardiomyocytes. Another possibility is that the cells were directed to the endoderm lineages, as exposure to these compounds was shown to severely disrupt mesoderm formation.

Embryotoxicity assays based on WEC have been invaluable in drug toxicology for decades, because they can study drug effects on whole systematic biological processes. Generally, WEC assays focus on drug toxicity on structural and morphological features, such as limb and appendage malformations, but suffer from species differences that can lead to inaccurate predictions in humans. In contrast, stem cell-based assays, including mEST and newly developed in vitro platforms using human pluripotent stem cells, offer a cheaper and less invasive method to measure drug toxicity on mammalian and human cell differentiation. However, they cannot characterize tissue morphogenesis and organ formation. Meaningful comparisons between these systems are difficult to draw due to significant variations in characterization and measurement readouts from each model. In a comparison of triazole exposure to rat WEC, zebrafish WEC and mEST, the zebrafish tests showed the best correlation, followed by mEST tests, regarding their toxicity levels relative to in vivo studies conducted in industry. Rat WEC had the lowest correlation scores, which was likely caused by differences in drug exposure times calculated for each system, illustrating the challenges in embryotoxicity model comparisons. Other studies on embryotoxicity indicated a comparable result between WEC and mEST models, but poor correlation with in vivo reports. Furthermore, these works suggest that a combination of different testing systems can provide better predictivity of embryotoxic potential. One study integrated mEST and zebrafish WEC to understand biological mechanisms of triclosan on early development, and found that triclosan causes developmental defects via disruption of pluripotent markers. In relation to the apparent heterogeneity of the cardiac organoids, cardiac organoids according to the present invention can serve as complementary tests to current well-established assays to assess teratogenicity in both cell differentiation and tissue morphogenesis, which can provide a comprehensive risk-assessment toolkit to better predict drug toxicity on fetal health.

The present invention is directed primarily to studying early developmental events and drug effects on embryonic cardiogenesis, instead of mimicking adult-like physiology and drug responses. Future experiments, such as lineage tracking, fate mapping, and single cell genomic sequencing of organoids at different developmental stages, can reveal parallelism between human cardiac development and cardiac organoid formation through comprehensive molecular evidence.

### Example

Experimental Procedures

Micropatterning of Tissue Culture Surfaces

Surface micropatterning on tissue culture polystyrene was carried out using the selective etching approach. Patterned wafers were (SU8 master) fabricated using standard SU8 photolithography to fabricate molds with raised features of patterns. Poly(dimethyl siloxane) (PDMS) prepared at a 10:1 wt/wt ratio of elastomer base to curing agent was casted onto SU8 masters and clamped down using clear transparency sheets and glass slides. This process produced thin PDMS stencils with clear-through holes from the raised patterns on the SU8 master molds. Non-fouling poly(ethylene glycol) (PEG) solution was prepared by combining 150 mg PEG 1000 (Polysciences, cat. no. 16666), 1.8 mL PEGDA 400 (Polysciences, cat. no. 01871), 14.55 mL isopropyl alcohol, and 0.45 mL MilliQ water. The solution was grafted onto 6-well tissue culture plates and cured under UV light exposure (Dymax UV Illuminator; model no. 2000EC) for 45 seconds. Micropatterns were fabricated by selective oxygen plasma etching (Oxygen plasma treatment system, PlasmaEtch PE50XL) of the PEG using the PDMS stencils. Micropatterned tissue culture plates were sterilized by immersing in 70% ethanol for 1 hour and subsequent washing with sterile phosphate buffered saline (PBS).

Cell Lines

Wild-type (WTC) hiPSC line was obtained from Dr. Conklin's laboratory at the Gladstone Institute of Cardiovascular Research. This hiPSC line was derived from a skin biopsy from a healthy adult Asian male donor in his early thirties. The original fibroblasts were reprogrammed using episomal methods with the factors of LIN28A, MYC (c-MYC), POU5F1 (OCT4) and SOX2. WTC GCaMPf6 hiPSC line was generated in Dr. Conklin's laboratory by targeting to the AAVS1 locus of WTC cells. A strong constitutive promoter (CAG) drives the expression of the GCaMP6f ORF. Yale-WT hiPSCs line was obtained from Dr. Abha Gupta's laboratory at the Yale University Department of Pediatrics and Child Study Center. Briefly hiPSCs were generated from the T-lymphocytes of a 25-year-old healthy South Asian male using the CytoTune-iPS Sendai Reprogramming kit.

Generation of Cardiac Organoids

Micropatterned surfaces were coated with diluted Geltrex hESC-qualified matrix (Life Technologies, cat. no. A1413302) at 37° C. for 1 hour prior to cell seeding. hiPSCs were cultured using standard PSC practices in Essential 8 (E8) medium (Life Technologies, cat. no. A1517001). At passaging confluency, cells were dissociated with Accutase (Life Technologies, cat. no. A1110501) and seeded at a density of 6.0×105 cells per well of the micropatterned 6-well plate (˜0.63×105 cells per cm2) supplemented with 10 μM Y27632 (BioVision, cat. no. 1784-5). Cardiac differentiation was initiated approximately 3 days after seeding (Day 0) when the micropatterns reached confluency, and performed via small molecule modulation of the Wnt/β-catenin pathway (Lian et al., 2012) with GSK3 inhibitor CHIR99021 (Day 0) (Stemgent, cat. no. 04-0004) and WNT pathway inhibitor IWP4 (Day 2) (Stemgent, cat. no. 04-0036). Small molecules were diluted in in RPMI 1640 medium (Life Technologies, cat. no. 11875093) supplemented with B27-minus insulin (RPMI/B27 minus insulin) (Life Technologies, cat. no. A1895601). Cardiac organoids began to contract around Day 9 of differentiation and were maintained in RPMI 1640 medium supplemented with complete B27 supplement (RPMI/B27 Complete) (Life Technologies cat. no. 17504044) until Day 20 for contractile and structural analysis.

Gene Expression Analysis

Gene expression was quantified using real-time qPCR analysis. On Day 20 of differentiation, cardiac organoids were sacrificed for RT-qPCR analysis. RNA was extracted using the RNeasy Mini Kit (Qiagen cat. no. 74104) and stored in −80° C. until needed. The RNA was then converted to cDNA using the Superscript IV First Strand Synthesis kit (Thermofisher cat. no. 18091050). Genes of interest includes cardiomyocyte-specific genes and stromal cell genes, plus TaqMan array of human factors for cardiogenesis (Thermofisher cat. no. 4414134). PCR plates were prepared and then run using the QuantStudio 3 Real-Time PCR System. All data was normalized to the respective housekeeping genes that were run in parallel with the rest of the gene assays. Value of DCt was calculated by subtracting the average Ct of housekeeping genes from the Ct of the genes of interest. Lower DCt indicates gene upregulation, where high DCt indicates gene downregulation.

Flow Cytometry Analysis

Cardiac organoids were dissociated using 0.25% Trypsin for 10-15 minutes. Cells were collected, centrifuged and washed with PBS. Cells were fixed and permeabilized with a mixture of 4% (vol/vol) paraformaldehyde and 0.2% (vol/vol) TritonX solution for 15 minutes. Cells were incubated with primary antibody cardiac troponin T (Thermofisher cat. no. MA5-12960) in a 1:250 dilution for 1 hour in PBS, and then incubated with AlexaFluor 546 secondary fluorophore for an additional hour. The cell suspension was washed, centrifuged and filtered through 35 μm mesh cell strainer. Flow cytometry was performed on the BDAccuriC6 at Flow Cytometry Core at Syracuse University.

Drug Treatment

Concentrations were chosen after evaluation of blood plasma concentrations reported for each drug from the FDA drug information database (accessdata.FDA.gov). Concentrations were chosen to be at or approximated by blood plasma concentrations, while accounting for drug solubility in water or DMSO, while also supplying a large range in order to detect potential toxicity. Drugs were diluted in the appropriate culture media at three concentrations each increasing by a factor of 10 with respective controls. Control samples were supplemented with water or DMSO (≤0.1%), depending on the solvent used to prepare the concentrated stock. Once initiated, the drugs were supplied continuously throughout the differentiation into cardiac organoids in order to mimic the continuous drug exposure during fetal development. Samples were terminated on Day 20 for motion tracking analysis and for fluorescence/confocal imaging to assess the developmental toxicity of specific drugs based on the organoid morphology and contractile physiology.

Analysis of Contraction Physiology

Organoids were imaged in an onstage microscope incubator (OkoLab Stage Top Incubator, UNO-T-HCO2) at 37° C. and 5% CO2 to maintain standard physiological conditions on a Nikon Ti-E inverted microscope with Andor Zyla 4.2+ digital CMOS camera. Videos of contracting cardiac organoids were recorded at 50 frames per second for ten seconds in brightfield and exported as a series of single frame image files. Contraction physiology was assessed using video-based motion tracking software that computes motion vectors based on block matching of pixel macroblocks from one frame to the next. The motion vectors were assimilated into a contraction motion waveform representative of contractile physiology, providing metrics such as contraction amplitude and frequency. Peak-to-peak interval is the time interval between contraction peak and relaxation peak. Contraction physiology was also assessed by recording the calcium transient using GCaMP6f hiPSC-derived cardiac organoids. Videos were taken under GFP excitation at 40 ms exposure time with 25 frames per second. Calcium flux signals were exported as Z-axis profiles in ImageJ. The fluorescence bleaching decay was corrected and time decay parameters τ0, τ50, τ75 were computed using in-house MATLAB scripts. The pulse duration is the time interval at which the calcium flux is at the half of the maximum flux. The time interval τ0 is defined as the time it takes for the calcium flux to reach peak fluorescence intensity, whereas τ50 and τ75 represent the time it takes for the calcium flux to decay 50% and 75% of the peak fluorescence, respectively. Relationships within the functional data was visualized utilizing R. Normalization to the zero mean, or Znormalization, was utilized to normalize and scale each parameter to have a mean of 0 with a range near 1. This preprocessing step ensures allows us to study the correlation and similarities of our studied variables. t-Stochastic Neighbor Embedding (t-SNE), an unsupervised machine learning algorithm, was used for exploratory data analysis of the impact of pattern sizes on the measured variables of the organoids. This modern dimensionality technique is able to take high-dimensional data and reduce multidimensional relationships between data to a lower dimensional space in such a way that similar relationships are grouped nearer to one another with a higher probability than dissimilar relationships or objects. This is accomplished by first creating a probability distribution of higher dimensional objects such that more similar pairs of higher dimensional objects are given a higher probability with more dissimilar points given a lower probability. A second probability distribution is then generated from this probability distribution for a lower dimensional map in such a way that preserves the maximum amount of similarity between the two probability distributions. t-SNE's ability to capture linear and nonlinear relationships between many variables makes it a powerful and versatile tool for investigating complex patterns while preserving higher dimensional structure of our data. t-SNE plots were generated using suggested parameters for perplexity, in order to condense the relationships between multiple recorded parameters down to a two-dimensional representative plot. Measurements were collected from mean values collected from 166 organoids. The actual t-SNE analysis was performed in R utilizing Jesse Krijthe's 2015 package Rtsne: T-Distrubed Stochastic Neighbor Embedding using a Barnes-Hut Implementation (https://github.com/jkrijthe/Rtsne) to reduce the representation of our parameters to two dimensions. Pattern diameters were displayed by varying size and color of each point, and then individual parameters were investigated by applying a color gradient in the t-SNE plots. A heatmap of the same data was generated to visualize each variables impact with respect to pattern diameter concurrently, while a correlogram gives further insight into the impact between parameters by utilizing Frank Harrell's Hmisc package to generate these figures https://CRAN.R-project.org/package=Hmi sc.

Immunofluorescence Staining and Confocal Microscopy

Organoids were characterized based on immunofluorescence staining patterns of cardiac tissue and smooth muscle-like tissue. After video recording, samples were sacrificed and fixed with 4% (vol/vol) paraformaldehyde (PFA) for 10 minutes. After PFA treatment, samples were washed and permeabilized with 0.2% (vol/vol) Triton X-100, blocked with 2% (wt/vol) bovine serum albumin (BSA) and incubated with the appropriate dilution of primary antibodies for 1 hour at room temperature. After incubation, the primary antibody was removed and washed with PBS. Secondary fluorescent antibodies were then incubated in the dark for 2 hours at appropriate dilutions and nuclei were tagged with 300 nM DAPI. All primary and secondary antibodies used are listed in Table 2 below. Confocal microscopy (Zeiss U880) was used to capture z-stacks (8 μm spacing between slices) of the organoids for height measurements and 3D reconstruction.

TABLE 2

| | | Catalog | |
|---|---|---|---|
| | Vendor | No. | Dilution |
| Primary antibodies (species) | | | |
| Cardiac troponin T (mouse) | Thermo Fisher | MS295P | 1:200 |
| | Scientific | | |
| Sarcomeric α-actinin (mouse) | Sigma-Aldrich | A7811 | 1:300 |
| Myosin heavy chain (mouse) | Abcam | Ab97715 | 1:200 |
| Cardiac troponin I (rabbit) | Abcam | Ab47003 | 1:200 |
| Vimentin (mouse) | Thermo Fisher | MA5-11883 | 1:100 |
| | Scientific | | |
| α-SM22 (rabbit) | Abcam | Ab14106 | 1:300 |
| Calponin (rabbit) | Abcam | Ab46794 | 1:200 |
| α-Smooth muscle actin (rabbit) | Abcam | Ab5694 | 1:100 |
| OCT4 (rabbit) | Abcam | Ab18976 | 1:200 |
| NANOG (mouse) | Thermo Fisher | MA1-017 | 1:100 |
| | Scientific | | |
| SSEA4 (mouse) | Stem Cell | 60062 | 1:200 |
| | Technologies | | |
| SOX2 (rabbit) | Thermo Fisher | PA1-094 | 1:100 |
| | Scientific | | |
| E-cadherin (mouse) | Abcam | Ab1416 | 1:200 |
| Secondary Antibodies | | | |
| Alexa Floor 488 goat anti-mouse | Thermo Fisher | A-11029 | 1:200 |
| | Scientific | | |
| Alexa Floor 546 goat anti-mouse | Thermo Fisher | A11003 | 1:200 |
| | Scientific | | |
| Alexa Floor 488 goat anti-rabbit | Thermo Fisher | A11008 | 1:200 |
| | Scientific | | |
| Alexa Floor 546 goat anti-rabbit | Thermo Fisher | A11010 | 1:200 |
| | Scientific | | |

Morphological and Structural Characterization of Cardiac Organoids

The cardiac organoids were assessed based on three parameters that characterize the overall cardiac tissue distribution and 3D morphology as seen in FIG. 3. All images were imported into ImageJ for image reconstruction and analysis. The Area Ratio was measured by using the circular or elliptical tool to approximate the area of fluorescence of tissue staining positive for cardiac tissue, and normalizing this area relative to the area of the entire pattern. The Height was measured by locating the top and bottom of the organoids using confocal microscopy. Lastly, the FWHM was determined by measuring the tissue diameter at half of the organoid height.

Zebrafish Whole Embryo Culture (zWEC) Embryotoxicity Assay

Transgenic Tg(my17:GFP) zebrafish that express GFP exclusively in cardiomyocytes were used to observe myocardium development in vivo. Adult fish were bred to generate a few hundred synchronized embryos, which were divided into individual wells of approximately 50 embryos. The drug stocks were diluted in zebrafish embryo medium. Chemicals at the same concentrations described in Table 1 were administered to chlorinated zebrafish embryos within the first 5 hpf, which is the estimated equivalent to the time point when the chemicals are introduced to the human cardiac organoids. Fresh embryo medium with chemicals is replaced at 24 hpf, when the embryos have developed a prominent linear heart tube, but not yet undergone looping. At 48 hpf, cardiac morphology and looping were scored as the first assessment of cardiac developmental toxicity on in vivo organogenesis. zWEC embryotoxic potentials of each chemical were scored based on the percentage of embryos exhibiting distinct cardiac morphology at 48 hpf. Normal looping (D-looping) refers to looping to the right-hand side of the embryo. Reverse looping (L-looping) is classified as looping towards the left side of the embryo, while no looping (N-looping) refers to a straight linear heart tube that has not successfully undergone cardiac looping events. A subset (˜20%) of embryos, including controls, did not express the GFP transgene, potentially due to silencing, and therefore cannot be classified as D/L/N-looping in this assay. Treatments that produced a rate of GFP absence that was significantly higher than controls are considered to reflect a severe abnormality in myocardial development.

Statistical Analysis

Data was plotted as box plots or mean±s.d. For single comparisons between two individual groups, a two-sided Student's t-test was used, and p≤0.05 was considered significant. For comparisons between more than two groups, one-way analysis of variance (ANOVA) was performed and p≤0.05 was considered significant. ANOVA analysis was supplemented with multiple comparison tests to determine significance between groups.

What is claimed is:

1. A method for screening a target compound for embryotoxicity, comprising the steps of:
forming an amount of cardiac organoids, each of which includes a contracting cardiomyocyte surrounded by a plurality of stromal cells distributed therearound, from a quantity of human induced pluripotent stem cells;
exposing the human induced pluripotent stem cells to the target compounds during the step of forming the amount of spatially organized cardiac organoids; and
evaluating an amount of cardiac differentiation, a contractile behavior, and a three dimensional tissue morphology of the amount of cardiac organoids after exposing the amount of spatially organized cardiac organoids to the target compounds to determine whether the target compound is embryotoxic.

2. The method of claim 1, wherein the cardiac organoids are spatially organized.

3. The method of claim 2, wherein the step of forming the amount of cardiac organoids is performed using a micropatterned substrate.

4. The method of claim 3, wherein the micropatterned substrate includes a plurality of circles each which has a diameter of between 200 and 1000 μm.

5. The method of claim 4, wherein the micropatterned substrate includes a plurality of circles each which has a diameter of 600 μm.

6. The method of claim 5, wherein the step of exposing the human induced pluripotent stem cells to the target compounds during the step of forming the amount of spatially organized cardiac organoids comprises exposing the human induced pluripotent stem cells on a first day of differentiation.

7. The method of claim 6, wherein the step of forming the amount of cardiac organoids includes the step of differentiating human induced pluripotent stem cells by small molecule modulation of the Wnt/β-catenin pathway.

8. The method of claim 7, wherein the step of forming the amount of cardiac organoids includes the step of oxygen plasma etching an amount of poly(ethylene glycol) to form the micropatterned substrate.

9. The method of claim 8, wherein the step of oxygen plasma etching the amount of poly(ethylene glycol) includes using a mask formed from poly(dimethyl siloxane).

10. The method of claim 9, wherein the step of forming the amount of cardiac organoids includes the step of coating the micropatterned surface were coated with a diluted hESC-qualified matrix.

11. The method of claim 10, wherein the step of forming the amount of cardiac organoids includes the step of differentiating the human induced pluripotent stem cells with a GSK3 inhibitor.

12. An organoid formed by differentiating a quantity of human induced pluripotent stem cells on a micropatterned substrate

13. The organoid of claim 12, wherein the micropatterned substrate includes a plurality of circles each which has a diameter of between 200 and 1000 μm.

14. The organoid of claim 13, wherein the micropatterned substrate includes a plurality of circles each which has a diameter of 600 μm.
